(19) United States
(12) Patent Application Publication
(10) Pub. No.: US 2026/0061039 A1
(43) Pub. Date: Mar. 5, 2026

(54) PRODUCTION OF 2-3-CYCLIC GMP-AMP (cGAMP) AND METHOD OF USE THEREOF

(71) Applicant: The Regents of the University of Colorado, a body corporate, Denver, CO (US)

(72) Inventors: Aaron T. Whiteley, Boulder, CO (US); Uday V. Tak, Westminster, CO (US); Philip J. Kranzusch, Brighton, MA (US); John J. Mekalanos, Newton, MA (US)

(21) Appl. No.: 19/286,185

(22) Filed: Jul. 30, 2025

Related U.S. Application Data
(63) Continuation-in-part of application No. PCT/US24/16988, filed on Feb. 23, 2024.
(60) Provisional application No. 63/486,766, filed on Feb. 24, 2023.

(51) Int. Cl. A61K 38/48; A61K 35/00; A61K 35/742; C12N 9/48; C12N 11/16

(52) U.S. Cl. CPC A61K 38/48; A61K 35/742; C12N 9/48; C12N 11/16; A61K 2035/115

(57) ABSTRACT

Systems, methods, and compositions for the synthesis of 2′3′-cGAMP.

### CROSS-REFERENCE TO RELATED APPLICATIONS

This application is a bypass continuation-in-part of PCT Application No. PCT/US2024/016988 having an international filing date of Feb. 23, 2024, which designated the United States, which PCT application claims the benefit of and priority to U.S. Provisional Application No. 63/486,766 filed Feb. 24, 2023, the specification, claims and drawings of which are incorporated herein by reference in their entirety.

### STATEMENT OF GOVERNMENT INTEREST

This invention was made with government support under grant number AT012346 awarded by the National Institutes of Health. The government retains certain rights in this invention.

### SEQUENCE LISTING

The instant application contains contents of the electronic sequence listing (90245.00971-Sequence Listing.xml; Size: 8,843 bytes; and Date of Creation: Feb. 22, 2024) is herein incorporated by reference in its entirety.

### TECHNICAL FIELD

The inventive technology is directed to the field of chemical synthesis, and in particular novel system, methods, and compositions for the in vivo production of 2′3′-Cyclic-GMP-AMP (2′3′-cGAMP).

### BACKGROUND

The human cGAS-cGAMP-STING pathway links detection of DNA in the cytoplasm with a potent antimicrobial and anticancer immune response. When cGAS binds DNA localized outside the nucleus it synthesizes the nucleotide second messenger 2′3′-cGAMP, which diffuses throughout the cell. 2′3′-cGAMP, also referred to herein as cGAMP, binds STING which activates a signaling cascade resulting in type I interferon activation, NF-κB-mediated inflammation, autophagy, and cell death. STING signaling was discovered in the context of infection, where this pathway links detection of pathogen DNA to antiviral and antibacterial signaling. However, the cGAS-cGAMP-STING pathway has recently gained attention for its pivotal role in anticancer signaling. Mice deficient in either cGAS or STING are unable to mount anticancer immune responses. In addition, the activity of some anticancer therapeutics, such as immune checkpoint inhibitors, are dependent on cGAS and STING. These findings spotlight the important role of cGAS-cGAMP-STING in disease.

Cyclic dinucleotides, like 2′3′-cGAMP are enzymatically synthesized by joining two nucleotide triphosphates (in this case from ATP and GTP). The phosphodiester bonds linking the nucleotides can be either 3′-5′ linked such as in RNA and DNA, or 2′-5′ linked, a comparatively rare bond in biology. 2′3′-cGAMP is asymmetric with one 2′ bond and one 3′ bond, giving the name 2′3′-cGAMP. Nucleotides are ideal second messengers because they are synthesized from abundant precursor NTPs and rapidly alter cellular signaling by binding specific receptors (e.g. STING). Given four abundant ribonucleotide triphosphates and different phosphodiester bonds, many cyclic dinucleotides are possible. Cyclic dinucleotides like 2′3′-cGAMP are also highly stable, which enables both intracellular signaling and uptake of these molecules from the extracellular space. 2′3′-cGAMP was discovered in 2013 as the first and only cyclic dinucleotide in mammalian cells.

As such, there exists a long-felt need for a simple, inexpensive, and effective system biosynthesize 2′3′-cGAMP independent of cGAS-directed synthesis, which can further be isolated and used as a therapeutic compound, such as a STING agonist. There furthers exists a long-felt need for a host cell, such as a bacterial probiotic or other similar host, that can be genetically engineered to biosynthesize 2′3′-cGAMP. This host cell can be administered to a subject in need thereof, and biosynthesize and deliver a therapeutically effective amount 2′3′-cGAMP to the subject and thereby activate the STING-pathway in vivo.

### SUMMARY OF THE INVENTION

The preset invention is directed to systems, methods, and composition for the synthesis of 2′3′-cGAMP. In one aspect, synthesis of 2′3′-cGAMP is through the action of one or more proteins generally referred to herein as a nucleotidyltransferase 5 (NTase05 or CD-NTase) enzyme.

In one aspect, a protein comprising an amino acid sequence having at least 70% sequence identity with any one of the NTase05 amino acid sequences listed in Table 1 catalyzes the production of 2′3′-cGAMP from its constituents ATP and GTP components.

In one aspect, the production of 2′3′-cGAMP from its constituents ATP and GTP components independent of the action of cGAS or in response to cytosolic DNA stimulation.

In certain aspects, the NTase05 enzyme of the invention can include a modified polypeptide, and may further be selected from the sequences according to SEQ ID NOs. 1-6, or a biologically active fragment thereof.

In another aspect, a NTase05 protein of the invention includes a modified peptide having a heterologous portion selected from the group consisting of: a signal peptide, a peptide tag, a dimerization domain, an oligomerization domain, an antibody, or an antibody fragment. In still further embodiments the peptide tag is a thioredoxin, Maltose-binding protein (MBP), SUMO2, Glutathione-S-Transferase (GST), calmodulin binding protein (CBP), protein C tag, Myc-tag, Halo-Tag, HA tag, Flag tag, His tag, biotin lag, V5 tag, or OmpA signal sequence tag. In still another embodiment, the antibody fragment is an Fc domain. In yet another embodiment, the polypeptide is immobilized on an object selected from the group consisting of a cell, a metal, a resin, a polymer, a ceramic, a glass, a microelectrode, a graphitic particle, a bead, a gel, a plate, an array, and a capillary tube.

In yet another aspect, an isolated nucleic acid molecule comprising a nucleotide sequence, which is complementary to a nucleic acid sequence described herein, is provided. In another aspect, a vector, such as an expression vector, comprising a nucleic acid molecule described herein, is provided.

In still another aspect, a host cell transfected, with an expression vector described herein, is provided.

In yet another aspect, a method of producing a polypeptide described herein, comprising culturing a host cell described herein in an appropriate culture medium to, thereby, producing the polypeptide, is provided.

In yet another aspect, a method of producing a 2′3′-cGAMP is described herein, comprising culturing a host cell described herein in an appropriate culture medium to, thereby, producing the polypeptide, which in turn biosynthesizes 2′3′-cGAMP, which can further be isolated and administered as a STING agonist, preferably as a pharmaceutical composition, and more preferably to a subject in need thereof.

In still another aspect, a non-human animal model engineered to express a polypeptide described herein, is provided. In one embodiment, the polypeptide is overexpressed. In another embodiment, the animal is a knock-in or a transgenic animal. In still another embodiment, the animal is a rodent.

In yet another aspect, a method of synthesizing nucleotides, and preferably 2′3′-cGAMP, comprising contacting a polypeptide described herein, or biologically active fragment thereof, with nucleotide substrates in vitro, ex vivo, or in vivo.

In yet another aspect, a method of producing a 2′3′-cGAMP is described herein, comprising culturing a host cell described herein in an appropriate culture medium to, thereby, producing the polypeptide, which in turn biosynthesizes 2′3′-cGAMP, wherein the host cell can be administered preferably as a pharmaceutical or probiotic composition, and more preferably to a subject in need thereof, wherein the 2′3′-cGAMP by the host cell acts as a STRONG agonist.

Additional aspects of the inventive technology will be evident from the detailed description and figures presented below.

### BRIEF DESCRIPTION OF THE FIGURES

FIG. 1A-G. Forward biochemical screen to identify bacterial 2′,3′-cGAMP synthases. (A) PEI-cellulose TLC analysis of enzyme reaction products produced when the indicated CD-NTases were incubated with α³²P-radiolabelled NTPs. Vibrio cholerae DncV was used to produce 3′,3′-cGAMP in lane one. Catalytically dead (CD) DncV^{DID79AIA}; inorganic phosphate (Pᵢ); origin (Ori.). Images are representative of n=2 separate experiments. (B) Analysis as in (A) after treatment with nuclease P1 (P1), which cleaves 3′-5′ phosphodiester bonds. Spots remaining after nuclease P1 treatment contain non-3′-5′ linked ³²P, such as 2′-5′ linkages. (C) Chemical structure of 3′,3′-cGAMP and schematic for P1 treatment and cleavage of the 3′-5′ linked phosphodiester bond. (D) Chemical structure of 2′,3′-cGAMP and schematic for P1 or Poxin treatment which hydrolyzes the 3′-5′ linked phosphodiester bond leaving the linear 2′-5′ linked linear product. (E) Phylogenetic analysis of CdnB02 homologues using DaCdnB as a seed. Abbreviations for bacterial species analyzed in this Application are provided in parentheses. (F) ELISA quantification of the indicated synthetic nucleotide standards at 50 nM used as a control for (G). Stars indicate significance determined after one-way ANOVA with Dunnett's multiple comparison test with **=P<0.05. Limit of detection (LOD) was calculated as the maximum signal produced by a non-specific nucleotide, indicated by the dashed line. (G) ELISA quantification of 2′,3′-cGAMP from enzyme reaction products produced when the indicated synthase is incubated with ATP and GTP. Where indicated, reaction products were further treated with VacV poxin. LOD was determined as in (F) Data shown are mean±standard error of the mean (SEM) from representative of n=2 biological replicates. (−) indicates buffer+ATP/GTP with no enzyme added. Asterisks indicate significance determined after a paired t-test for reaction vs poxin-treated samples, with *=P<0.05.

FIG. 2A-M. A CdnB-Cap14 CBASS system signals via 2′,3′-cGAMP to restrict phage replication. (A) Type I CBASS operon from B. thuringiensis Bt407 which encodes the CdnB02 synthase and an adjacent Saf2™-SAVED/Cap14 effector. (B) Indicated operons were cloned into B. subtilis PY79 as an expression host into the amyE locus and consist of an empty vector (EV), the wild type BtCBASS system (referred to as BtCBASS), and BtCBASS^{CD} which expresses a catalytically dead (CD) version of CdnB^{D77A/D79A} effectively acting as a cGAMP-null variant. (C) Double agar overlay assay of B. subtilis expressing chromosomally integrated EV, BtCBASS, or BtCBASS^{CD} challenged with serial dilutions of phage SPP1, phiB002 (D), phi29 (E), or SPβ (F) Data are representative images from n=3 biological replicates. Quantification represents plaque-forming units/ml (PFU/ml) and are from n=3 biological replicates. LOD=limit of detection. (G) Growth of B. subtilis expressing the indicated systems in liquid culture measured by optical density (OD₆₀₀) after infection with phage SPP1 at the indicated multiplicity of infection (MOI). A representative growth curve is shown from n>3 experiments with identical results. (H) Time course of SPP1 replication during liquid culture infection of B. subtilis expressing the indicated systems at the indicated MOIs (G). Shown are the plaque-forming units per milliliter (PFU/ml) at the indicated time points after post-infection. Data represents the mean±SEM of n=3 biological replicates. (I) Fluorescence microscopy of B. subtilis strains expressing the indicated systems after liquid-culture infection with SPP1 at an MOI of 4 in 30-minute intervals (related to 2G and H). The bacterial membrane was visualized with the dye FM143x (green), and propidium iodide (false-colored in magenta) was used to probe membrane disruption. Images are from a single experiment, representative of >10 fields of view per condition, and was repeated 3 times with similar results. (J) Related to (2G, H, I): representative zoomed-in fields of view showing PI uptake and membrane damage at 60- and 90-minutes post-infection with SPP1 for B. subtilis expressing CBASS vs CBASS^{CD}. (K) Additional fields of view for B. subtilis expressing CBASS at 60 minutes post infection highlighting membrane disruption, PI uptake, and cellular morphology. (L) Percentage of PI+ cells at 60 minutes post-infection from (I). Data represent mean±SEM from 18 fields of view analyzed from n=3 biological replicates (6 fields of view/replicate). Stars indicate significance after one-way ANOVA with Dunnet's multiple comparisons test with **=P<0.05. (M) ELISA quantification of 2′,3′-cGAMP from cell lysates of the indicated B. subtilis strains harvested at 60 minutes post-infection with SPP1, phiB002 at an MOI of 4, or uninfected. Mean±SEM from n=3 replicates. Stars indicate significance after one-way ANOVA with Dunnet's multiple comparisons test with **=P<0.05.

FIG. 3A-K. The Cap14 effector binds 2′,3′-cGAMP and forms oligomers. (A) Domain architecture of BtCap14 with Saf-2™ and SAVED domain organization by amino acid (AA). (B) AlphaFold-3 model of BtCap14 monomer with delineation of the N-terminal Saf-2™ domain, membrane boundaries, and globular C-terminal SAVED domain. (C) Binding of cGAMP isoforms to recombinant BtCap14_{6×his} via microscale thermophoresis. Data represent mean±SEM from n=3 runs. ND=not detectable. (D) Representative Cryo-EM 2D class averages of recombinant BtCap14 in amphipols with and without 2′,3′-cGAMP. (E) Cryo-EM reconstruction of the BtCap14 SAVED domain bound to 2′,3′-cGAMP. Colors represent the individual SAVED domains, with the same color indicating protomeric units formed by the dimer. The plane of the membrane is shown in respect to the globular SAVED domain and the white outline represents disordered density for the Saf-2™ domain. (F) Atomic model of the BtCap14 SAVED domain with individual 2′,3′-cGAMP molecules highlighted at each interface. (G-H) An individual 2′,3′-cGAMP density and model fit with binding site residues identified. The orientation of AMP and GMP are indicated in black. (I) Binding of 2′,3′-cGAMP to BtCap14_{6×his} and binding-site substitution variants by microscale thermophoresis. ND=not detectable. (J) Effect of Cap14 binding variants on BtCBASS phage protection. Double agar overlay assay of B. subtilis expressing the indicated BtCBASS operons expressing Cap14 binding variants challenged with phage SPP1 and representative quantification of PFU/ml. n=3 biological replicates. LOD=limit of detection. (K) Cap14 binding variants do not disrupt protein expression. Immunoblot showing expression levels of the indicated Cap14-HA fusion proteins characterized in (J) in B. subtilis PY79 with RNAP as a loading control. The first lane represents the CBASS operon without tags as a negative control for antibody background. Shown is the monomeric band of Cap14 at ˜42 kDa.

FIG. 3A-K. The Cap14 effector binds 2′,3′-cGAMP and forms oligomers. (A) Domain architecture of BtCap14 with Saf-2™ and SAVED domain organization by amino acid (AA). (B) AlphaFold-3 model of BtCap14 monomer with delineation of the N-terminal Saf-2™ domain, membrane boundaries, and globular C-terminal SAVED domain. (C) Binding of cGAMP isoforms to recombinant BtCap14_{6×his} via microscale thermophoresis. Data represent mean±SEM from n=3 runs. ND=not detectable. (D) Representative Cryo-EM 2D class averages of recombinant BtCap14 in amphipols with and without 2′,3′-cGAMP. (E) Cryo-EM reconstruction of the BtCap14 SAVED domain bound to 2′,3′-cGAMP. Colors represent the individual SAVED domains, with the same color indicating protomeric units formed by the dimer. The plane of the membrane is shown in respect to the globular SAVED domain and the white outline represents disordered density for the Saf-2™ domain. (F) Atomic model of the BtCap14 SAVED domain with individual 2′,3′-cGAMP molecules highlighted at each interface. (G-H) An individual 2′,3′-cGAMP density and model fit with binding site residues identified. The orientation of AMP and GMP are indicated in black. (I) Binding of 2′,3′-cGAMP to BtCap14_{6×his} and binding-site substitution variants by microscale thermophoresis. ND=not detectable. (J) Effect of Cap14 binding variants on BtCBASS phage protection. Double agar overlay assay of B. subtilis expressing the indicated BtCBASS operons expressing Cap14 binding variants challenged with phage SPP1 and representative quantification of PFU/ml. n=3 biological replicates. LOD=limit of detection. (K) Cap14 binding variants do not disrupt protein expression. Immunoblot showing expression levels of the indicated Cap14-HA fusion proteins characterized in (J) in B. subtilis PY79 with RNAP as a loading control. The first lane represents the CBASS operon without tags as a negative control for antibody background. Shown is the monomeric band of Cap14 at ˜42 kDa.

FIG. 4A-G. Cap14 forms cGAMP-gated channels and depolarizes bacterial membranes. (A) Representative current-trace recordings of BtCap14_{6×his} in planar lipid bilayers with or without 1 μM 2′,3′-cGAMP added to the cis side of the bilayer. Recordings were collected at a voltage clamp of +40 mV. Data are representative of n>10 recordings from >3 separate preparations of protein. (B) Current/voltage relationship (I/V) of BtCap14+vehicle control or 1 μM 2′,3′-cGAMP, and recombinant binding variants Y345A and K213E. Data is the mean±standard deviation (SD) for a representative experiment from n≥3 separate experiments with similar results. (C) I/V relationship of BtCap14+1 μM 2′,3′-cGAMP with the indicated salts (IM) substituted in a recording solution of 10 mM HEPES pH 7.2: sodium chloride (NaCl), Tetraethylammonium-chloride (TEA-Cl), or potassium gluconate (K-Gluc) as the recording solution under symmetric conditions. Data is the mean±SD for a representative experiment from n≥3 separate experiments. (D) Membrane depolarization of B. subtilis expressing CBASS vs CBASS^{CD} at the indicated timepoints post-infection with phage SPP1 at an MOI of 4 measuring Disc3 (5) dye release into the membrane. Shown is the fold-change of Disc3 (5) fluorescence indicating release into the medium normalized to the value for uninfected cells at each timepoint. Data represents SEM from n=9 technical replicates from n=3 biological replicates. Stars indicate significance after two-tailed Mann-Whitney test, ns=not significant, **=P<0.05. (E) Double-agar overlay assay of SPP1 challenge on B. subtilis expressing BtCBASS with the indicated mutations in the Saf-2™ region and the linker between the SAVED domain in PFU/ml. (F) AlphaFold-3 model of a BtCap14 dimer with the indicated residues highlighted in (H) that disrupted expression, stability (K103A, K105A), and/or resulted in an intermediate phage protection phenotype (D54K). (G) AlphaFold-3 multimer prediction of BtCap14 6mer assembly highlighting the predicted pore opening vs the D54K 6mer assembly demonstrating constriction of the Saf2™ pore domain.

FIG. 5A-K. Membranolytic cell death is conserved in 2™-STING-mediated CBASS immunity. (A) Operon structure of a Type I CBASS system from Flavobacterium sp. (FsCBASS) that contains a CdnE synthase and a 2™-STING/Cap13 effector. The predicted domain architecture for Cap13 is displayed below. (B) Double agar overlay assay of B. subtilis expressing chromosomally integrated EV, or FsCBASS challenged with serial dilutions of phage SPP1. Data are representative images from n=3 biological replicates. Quantification represents plaque-forming units/ml (PFU/ml) and are from n=3 biological replicates. LOD=limit of detection. (C-E) Same as (5B) but with phages phiB002, phi29, and SPB respectively. (F) Growth of B. subtilis expressing the indicated systems in liquid culture measured by optical density (OD₆₀₀) after infection with phage SPP1 at the indicated multiplicity of infection (MOI). A representative growth curve is shown from n>3 experiments with identical results. (G) Time course of SPP1 replication during liquid culture infection of B. subtilis expressing the indicated systems at the indicated MOIs from (5F). Shown are the plaque-forming units per milliliter (PFU/ml) at the indicated time points after post-infection. Data represents the mean±SEM of n=3 biological replicates. (H) Fluorescence microscopy of B. subtilis strains expressing EV or FsCBASS after liquid-culture infection with SPP1 at an MOI of 4 in 30-minute intervals (related to 5F-G). The bacterial membrane was visualized with the dye FM143x (green), and propidium iodide (false-colored in magenta) was used to probe membrane disruption. Images are from a single experiment, representative of >10 fields of view per condition, and were repeated 3 times with similar results. (I) Related to (5H) representative additional images showing PI uptake, cellular morphology, and membrane damage of B. subtilis expressing EV or FsCBASS at 60 minutes post-infection. (K) Percentage of PI+ cells at 60 minutes post-infection from (I). Data represent mean±SEM from 18 fields of view analyzed from n=3 biological replicates (6 fields of view/replicate). Stars indicate significance after one-way ANOVA with Dunnet's multiple comparisons test with **=P<0.05.

FIG. 6A-F. Domain-swapping of CBASS effectors enables engineering of a 2′,3′-cGAMP biosensor. (A) Receptor-swapping strategy for engineering a 2′,3′-cGAMP-activated biosensor. GbCap5 (Geobacillus) contains an N-terminal HNH endonuclease with a C-terminal SAVED domain that responds to 3′,2′-cGAMP and 2′,3′-cGAMP at 1 μM (data not shown). The SAVED domain from Clostridium botulinum (CbCap14) was swapped with the GbSAVED domain to construct the chimeric nuclease effector: Chimera10. Numbers represent amino acid positions. (B) cGAMP-dependent nuclease activity of Chimera10. Linear DS DNA was incubated with Chimera10 and 1 μM of the indicated cyclic dinucleotide for 30 minutes at 37° C. Reactions were separated by agarose gel electrophoresis. Data are representative of n>3 separate experiments. (C) Same assay as (B), but using the indicated concentration of synthetic 2′,3′-cGAMP. Data are representative of n>3 separate experiments. (D) Fluorescence-based DNase activity assay of Chimera10 activity using DNAse Alert using the indicated synthetic nucleotide second messengers. Data is represented as fold change/vehicle control (Chimera 10 in buffer, no nucleotides). (E) Same as (D) but with the indicated amounts of 2′,3′-cGAMP. (F) Detection of 2′,3′-cGAMP from bacterial cell lysates using Chimera10. Cell lysates from B. subtilis expressing BtCBASS or BtCBASS^{CD} (cGAMP null) were prepared after infection with SPP1 or phiB002 at an MOI of 4 for 50 minutes. Data was normalized as fold-change over the cGAMP null strain

FIG. 7. Model for cell death by bacterial TM effectors in CBASS immunity. Model for Cap14 and Cap13 mediated cell death. Phage infection triggers activation of the CD-NTase through an as-of-yet unknown trigger and results in cyclic oligonucleotide production. CDNs bind to Cap14 which initiates oligomerization, ion flux, and ultimately gross cell lysis which inhibits viral replication.

FIG. 8A-E. Bacterial CdnB02 enzymes and their products. (A) PEI-cellulose TLC analysis of CD-NTase reaction products produced when CD-NTase005 was incubated with the indicated α³²P-radiolabelled and unlabeled NTPs. (B) PEI-cellulose TLC analysis of enzyme reaction products of DaCdnB02 and the indicated homologues Mn²⁺ added at either 0, 0.01, 0.1, or 1 mM. The approximate migration of 2′,3′-cGAMP and linear intermediate pppG(2′,5′)pA are indicated with arrows. (C) Enzymatic reaction catalyzed by human cGAS displaying formation of the linear intermediate and the final 2′3′-cGAMP product. Reproduced with permission from Whiteley et al., Nature 2019. (D) Cladogram of CD-NTases as first described by Whiteley et al., Nature 2019 with CdnB02 clade highlighted. Additional clades for which a validated cyclic oligonucleotide product has been experimentally confirmed are indicated with their product. (E) Coomassie-stained SDS-PAGE gel indicating the purity of recombinant enzymes used in this Application. The approximate molecular weights are: 6×his-MBP-hcGAS (˜85 kDa), BtCdnB (˜49 kDa), CbCdnB (˜55 kDa), and 6×his-SUMO-VacV poxin (˜25 kDa). It is unclear why CbCdnB migrates slower on the SDS-PAGE gel.

FIG. 9A-F. Purification and biochemical analysis of recombinant BtCap14. (A) Domain annotation of BtCap14 using TMHMM software. Shown is a probability plot of the amino acids predicted to be transmembrane or soluble with a cartoon diagram above. (B) Coomassie-stained SDS-PAGE gel indicating purity of recombinant full-length BtCap14 and binding variants used in this Application. The monomeric weight of BtCap14 is ˜42 kDa. (C) Gel filtration chromatogram indicating absorbance at 280 nm (A280) of BtCap14_{6×his} extracted from E. coli membranes in DDM micelles or exchanged into amphipol A8-35 on a Superdex 200 10/30 column in 20 mM sodium phosphate pH 7.2, 500 mM KCl (+0.05% DDM for detergent extracted sample), as compared to protein standards of known molecular weight. (D) Coomassie-stained SDS-PAGE gel indicating the purity of BtCap14 peaks isolated from DDM extraction or after amphipol exchange (* indicates the peak collected). The monomeric weight of BtCap14 is ˜42 kDa. (E) Representative micrograph of BtCap14 in A8-35 amphipols and representative 2D class averages showing various oligomeric assemblies. (F) Representative micrograph of BtCap14+2′,3′-cGAMP in A8-35 amphipols and representative 2D class averages.

FIG. 10A-J. Cryo-EM reconstruction of BtCap14 SAVED domain+2′,3′-cGAMP. (A) Cryo-EM data processing summary to obtain the BtCap14+2′,3′-cGAMP SAVED domain filament reconstruction. (B) Fourier shell correlation (FSC) of the final reconstruction from Cryosparc. (C) Orientation distribution map of final reconstruction from CryoSparc. (D) Local resolution map of the final density map. (E) Atomic model built into the final density map. (F) Map to model fit of individual chains indicated by amino acid number. The map was contoured at 0.2. (G) Surface electrostatic map of the BtCap14 SAVED structure from the front with the 2′,3′-cGAMP binding pocket highlighted. (H) Density for 2′,3′-cGAMP within a single monomer (chain A) contoured at 0.4. (I) Representative density of one 2′,3′-cGAMP molecule from (H). (J) Densities of 2′,3′-cGAMP binding site residues tested in this Application.

FIG. 11A-B. AlphaFold-3 modeling of BtCap14 pore assembly. (A) Overlay of an AlphaFold-3 prediction of full-length BtCap14 6mer (colored by PLDDT) with our experimentally determined structure of the SAVED domain (colored in light green) and density (grey). The Saf-2™ domain is modeled within the predicted membrane region. (B) AlphaFold-3 multimer predictions of BtCap14 assemblies colored by amino acid charge indicating the opening of a putative pore at higher assemblies.

FIG. 12A-I. Analysis of BtCap14 channel activity. (A) Schematic of horizontal planar lipid bilayer set up for voltage-clamp recordings. All proteins and cGAMP were added from the cis side (top chamber) of the bilayer. Recording buffer was a symmetric solution of 10 mM HEPES pH 7.2, 1 M KCl in the cis and trans compartments unless otherwise indicated. (B) Representative current trace of an open BtCap14 channel inhibited by 1 μM gadolinium chloride (Gd³⁺). Data are representative of n=3 separate runs with similar results. (C) Single channel recording of BtCap14+1 μM 2′,3′-cGAMP at +100 mV in the indicated recording solution. Data are representative of n≥3 separate experiments with similar results. (D) Representative current trace of BtCap14+2′,3′-cGAMP in 1 M K-Gluconate+10 mM KCl, and then buffer exchanged and perfused with 10 mM HEPES pH 7.2+1 M KCl on the cis side of the bilayer. Data are representative of n=3 separate runs with similar results. (E-F) Immunoblots of BtCap14_{HA} with the indicated mutations. RNAP serves as a loading control. In E, the “CBASS” only lane represents untagged Cap14 to verify antibody specificity against the HA tag. Data are representative of 3 separate experiments with similar results. (G) Double agar overlay assay of BtCBASS expressing BtCap14^{D54K} challenged with phage SPP1. (H) AlphaFold-3 monomer prediction of BtCap14 colored by PLDDT with residues in the Saf-2™ and SAVED linker region that resulted in disrupted protection and/or protein instability highlighted. (I) ClustalOmega alignment of closest BtCap14 homologues in the Saf-2™ region. Residues highlighted with an asterisk (*) resulted in disrupted protein stability/expression and/or intermediate phage protection.

FIG. 13A-B. AlphaFold modeling of Cap13. AlphaFold-3 modeling full-length FsSTING (Cap13) as a dimer shown from the front to side views colored by PLDDT. Inlet: bottom view of the 2™ domain colored by PLDDT and surface charge indicating a putative positively charged cavity at the center of the protein. (B) AlphaFold-3 modeling full-length FsSTING (Cap13) as a tetramer displayed as in (A). Inlet: bottom view of the assembly colored by PLDDT and surface charge, indicating assembly of a putative charged cavity in the 2™ domain.

FIG. 14A-B. Chimera 10 purification and nuclease assay description. (A) Coomassie stained SDS-PAGE gel demonstrating protein purity after expression and purification steps for 6×his-SUMO-Chimera10 (Final monomeric molecular weight after 6×-his-SUMO tag cleavage: ˜45 kDa). Representative of n>3 purifications with identical results. (B) Schematic for fluorescence-based nuclease reporter assay with Chimera 10 using the DNAseAlert substrate (IDT) based on the manufacturer's handbook.

FIG. 15. The human cGAS-cGAMP-STING pathway. Cyclic GMP-AMP Synthase (cGAS) binds double stranded DNA to synthesize 2′,3′-cyclic GMP-AMP (2′3′-cGAMP), activating Stimulator of Interferon Genes (STING). STING signaling is crucial for immune system and anticancer signaling.

FIGS. 16A-F. Novel discovery of bacterial CD-NTase enzymes that produce 2′,3′-cGAMP: (a) Thin layer chromatography (TLC) of cyclic oligonucleotides produced by diverse bacterial CD-NTase enzymes across all clades (reproduced from Whiteley et al., Nature 2019). (b) The arrow indicates treatment of the products with PI endonuclease, which specifically cleaves 3′-5′ linked phosphodiester bonds. This TLC demonstrates reaction products that were resistant to P1 cleavage indicating the presence of a 2′-5′ linked phosphodiester bond such as in 2′,3′-cGAMP. (c) Confirmation that CD-NTase005 (sometimes referred to as CD-NTase) uses ATP and GTP as substrates, whereas CD-NTase036 uses ATP exclusively, thus excluding 2′3′-cGAMP as its reaction product. Radiolabeled NTP precursors were used in CD-NTase reactions, and the product formed by CD-NTase005 uses ATP and GTP. (d) CD-NTase reactions using homologues of CD-NTase005 in the presence of increasing manganese. CD-NTase005-03 from Bacillus thuringiensis 407 forms a product that appears to be 2′,3′-cGAMP, accelerated by the presence of manganese. (e) 2′,3′-cGAMP specific ELISA (Arbor Assays) to confirm the identity of the reaction product. In this panel, Synthetic cGAMP from all four isoforms as well as linear 2′,5′-pppGpA, cyclic di-AMP, and cyclic di-GMP were tested for cross-reactivity as controls, only 2′,3′-cGAMP gives a detectable signal using this assay. (f) CD-NTase reaction products from CD-NTase005-03 (Bacillus thuringiensis (Bt) and CD-NTase005-05 (Clostridium botulinum (Cb)) were confirmed to produce 2′3′-cGAMP via the same ELISA assay described in 1e. Reactions were also treated with VacV poxin enzyme or PI endonuclease for one hour which convert 2′3′-cGAMP into a linear product which is not detected by the ELISA. In the poxin or P1-treated samples, the signal was significantly reduced compared to the untreated reaction, indicating that the product is 2′,3′-cGAMP. Recombinantly purified human cGAS which exclusively produces 2′,3′-cGAMP was used as a control. Like the bacterial CD-NTases 005-03 and 005-05, the product is 2′,3′-cGAMP which are sensitive to poxin and PI treatment.

FIG. 17. Measuring 2′3′-cGAMP in cellular lysates. Chimera 10 displays higher activity, reflecting a higher 2′3′-cGAMP concentration, in lysate derived from WT CdnB expressed during phage infection, as compared to a catalytically dead strain or an empty vector. The present inventors demonstrated the measurement of 2/3/-cGAMP in cellular lysates. Specifically, B. subtilis PY79 expressing Empty vector (EV), BtCBASS system, or BtCBASS CdnB-Catalytically dead were grown in 200 ml of LB+phage salts), and infected with phage SPP1 at an MOI of 2. Cells were harvested 40 minutes post infection, concentrated, and resuspended in 400 μl of water. Bacteria were lysed via bead beating, boiled for 20 minutes, and cellular debris was removed by centrifugation. The supernatant was used for measurement of 2′3′-cGAMP. The procedure include a reaction mixture including: 10 mM Tris-HCl, 25 mM KCl, 1 mM DTT, and 3 μg Chimera10. DNAsealert tube mixed with 5 ml total buffer master mix. Further, 10 μl of cell lysate (described above) was incubated with indicated conditions for 1 hour at 37° C. Chimera10 activity was measured by monitoring changes in relative fluorescence units (RFU) at the end of the reaction. Chimera 10 degrades a fluorescent DNA probe in this reaction in response to 2′3′-cGAMP. Again, as described in FIG. 17, Chimera10 displays higher activity, reflecting a higher 2′3′-cGAMP concentration, in lysate derived from WT CdnB expressed during phage infection, as compared to a catalytically dead strain or an empty vector. (Applicant specifically incorporates by reference 63/447,555 filed Feb. 22, 2023, with respect to the production, and methods of using chimeric biosensor peptides configured to detect and quantifying 2′3′-Cyclic-GMP-AMP (2′3′ cGAMP)).

### DETAILED DESCRIPTION OF THE INVENTION

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries.

In one embodiment, the invention includes a wild-type or modified polypeptide that catalyzes synthesis of 2′3′-Cyclic-GMP-AMP (2′3′-cGAMP), wherein said modified polypeptide comprises an amino acid sequence having at least 70% identity to any one of the amino acid sequences according to SEQ ID NOs. 1-6, or a biologically active fragment thereof. The modified polypeptide can catalyze production of nucleotides in the absence of a ligand, such as double-stranded DNA, and preferably double-stranded DNA is located in the cytoplasm of a cell. In this manner,

In certain embodiments, 2′3′-cGAMP can be synthesized, for example in vitro, or preferably in a host cell. In this latter embodiment, the 2′3′-cGAMP can be synthesized independent of the action of cyclic GMP-AMP synthase (cGAS), and can further be secreted from the host cell and delivered to a cell of a subject, such as a human or animal, and act as an agonist of the Stimulator of Interferon Genes (STING) receptor. In this manner, activation of STING in a subject occurs independent of cGAS, and without the presence or activity of cGAS.

In one embodiment, the invention includes a wild-type or modified polypeptide that catalyzes synthesis of 2′3′-cGAMP according to SEQ ID NO. 1-6, or a fragment thereof, wherein the polypeptide further comprising a heterologous polypeptide. In this embodiment, the heterologous polypeptide of the invention is selected from the group consisting of: a signal peptide, a peptide tag, a dimerization domain, an oligomerization domain, an antibody, or an antibody fragment, such as an Fc domain. In still further embodiments, the peptide tag of the invention is a thioredoxra. Maltose-binding protein (MBP), SUMO2. Ghrtathione-S-Transferase (GST), calmodulin binding protein (CBP), protein C tag. Myc tag. HaloTag, HA tag, Flag tag, His tag, biotin tag, V5 tag, or OmpA signal sequence tag.

In still further embodiments, the invention includes a nucleic acid molecule, and preferably an isolated nucleotide sequence, encoding the polypeptide wild-type or modified polypeptide that catalyzes synthesis of 2′3′-cGAMP according to SEQ ID NO. 1-6, or a fragment thereof. In an alternative embodiment, the invention includes a nucleic acid molecule, and preferably an isolated nucleotide sequence, encoding the polypeptide wild-type or modified polypeptide that catalyzes synthesis of 2′3′-cGAMP according to SEQ ID NO. 1-6, or a fragment thereof, that is operably linked to a promoter forming an expression vector that can be used to transform a host cell, such as bacterial cell or a eukaryotic cell, and preferably a probiotic enteric bacterial cell. In additional embodiments, the host cell appropriate culture medium to, thereby, produce the polypeptide of the invention and/or 2′3′-cGAMP, which can further be isolated from the medium or host cell.

In still further embodiments, the invention includes a wild-type or modified polypeptide that catalyzes synthesis of 2′3′-2′3′-cGAMP according to SEQ ID NO. 1-6, or a fragment thereof, and a pharmaceutically acceptable carrier, which can be selected from the group consisting of excipients, and/or diluents. In alternative embodiments, the invention includes a pharmaceutical composition comprising the 2′3′-cGAMP isolated from a host cell, and a pharmaceutically acceptable carrier.

Additional embodiments may include methods of treating a disease or condition, and preferably a disease or condition that is treatable through the administration of a STING agonist, the method comprising, administering a therapeutically effective amount of the pharmaceutical composition of the invention to a subject in need thereof, wherein the 2′3′-cGAMP activates the STING receptor in the subject.

Additional embodiments may include methods of treating a disease or condition, and preferably a disease or condition that is treatable through the administration of a STING agonist, the method comprising, administering a therapeutically effective amount of the pharmaceutical composition of the invention to a subject in need thereof, wherein the 2′3′-cGAMP activates the STING receptor in the subject.

In still further embodiments, the invention includes a pharmaceutical composition comprising a host cell, and preferably a bacterial host cell, expressing a wild-type or modified CD-NTase polypeptide that catalyzes synthesis of 2′3′-cGAMP, or a fragment thereof, and a pharmaceutically acceptable carrier, which can be selected from the group consisting of excipients, and/or diluents. In still further embodiments, the invention includes a pharmaceutical composition comprising a host cell expressing a wild-type or modified polypeptide that catalyzes synthesis of 2′3′-cGAMP according to SEQ ID NO. 1-6, or a fragment thereof, and a pharmaceutically acceptable carrier, which can be selected from the group consisting of excipients, and/or diluents.

Additional embodiments may include methods of treating a disease or condition, and preferably a disease or condition that is treatable through the administration of a STING agonist, the method comprising, administering a therapeutically effective amount of the pharmaceutical composition of the invention to a subject in need thereof, wherein the 2′3′-cGAMP activates the STING receptor in the subject.

Additional embodiment include a non-human animal model, such as a rodent, engineered to express a wild-type or modified polypeptide that catalyzes synthesis of 2′3′-cGAMP according to SEQ ID NO. 1-6, or a fragment thereof. In certain embodiments, the polypeptide is overexpressed in the non-human animal model, which can further include a knock-in or a transgenic animal. In a preferred embodiment, the expression of cGAS has been disrupted or knocked-out.

Further embodiment include a host cell, and preferably a bacterial cell or a eukaryotic cell, and more preferably a probiotic bacterial cell, which is genetically modified to express a nucleotide sequence encoding heterologous CD-NTase polypeptide that catalyzes biosynthesis of 2′3′-cGAMP, or a biologically active fragment thereof. Further embodiment include a host cell, and preferably a bacterial cell or a eukaryotic cell, and more preferably a probiotic bacterial cell, which is genetically modified to express a nucleotide sequence encoding heterologous polypeptide that catalyzes biosynthesis of 2′3′-cGAMP, wherein said polypeptide comprises an amino acid sequence having at least 70% identity to any one of the amino acid sequences according to SEQ ID NOs. 1-6, or a biologically active fragment thereof.

Additional embodiment of the invention include method of synthesizing 2′3′-cGAMP comprising the step of contacting one or more CD-NTase polypeptides of the invention that catalyzes biosynthesis of 2′3′-cGAMP, or a biologically active fragment thereof, with the 2′3′-cGAMP nucleotide substrates adenosine triphosphate (ATP), and guanosine-5′-triphosphate (GTP). Synthesis of 2′3′-cGAMP can occur in vitro, in vivo, or ex vitro as described herein.

Additional embodiments of the invention are directed to isolated nucleic acid molecules that encode a wild-type, or modified polypeptide that catalyzes production of nucleotide-based second messengers, and preferably 2′3′-cGAMP, wherein said polypeptide comprises an amino acid sequence having at least 70% identity to any one of the amino acid sequences for the nucleotidyltransferase proteins (CD-NTase) listed in Table 1 (SEQ ID NO. 1), or derived from the bacterial strains identified in Table 1. As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules winch are present in the natural source of the nucleic acid. Preferably, an “isolated” nucleic acid is free of sequences which naturally flank the nucleic acid (i.e., sequences located at the 5 and 3′ ends of the nucleic acid) in the genomic DNA of the organism from which the nucleic acid is derived. For example, in various embodiments, the isolated nucleic acid molecule that encodes a modified CD-NTase polypeptide, or biologically active portions thereof, can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule that encodes a modified CD-NTase polypeptide, and preferably a CD-NTase polypeptide according to SEQ ID NOs. 1-6, or biologically active portions thereof, encompassed by the present invention, can include a nucleotide sequence encoding the same which is at least about 50%, preferably at least about 60%, more preferably at least about 70%, yet more preferably at least about 80%, still more preferably at least about 90%, and most preferably at least about 95% or more (e.g., about 98%) homologous to a sequence encoding a CD-NTase polypeptide according to SEQ ID NOs. 1-6. For example, a modified CD-NTase polypeptide cDNA can be isolated from a bacterium using all or portion of the nucleotide sequence encoding a CD-NTase polypeptide according to SEQ ID NOs. 1-6, or fragment thereof.

Portions of proteins encoded by the modified CD-NTase nucleic acid molecule encompassed by the present invention are preferably biologically active portions of the modified CD-NTase polypeptide, and preferably a CD-NTase polypeptide according to SEQ ID NOs. 1-6, or fragment thereof. As used herein, the term “biologically active portion” of the modified CD-NTase polypeptide is intended to include a portion, e.g., a domain/motif of the modified CD-NTase polypeptide that has one or more of the biological activities of the full-length modified CD-NTase polypeptide, respectively. The invention further encompasses nucleic acid molecules that differ from the nucleotide sequences shown in Table 1, or fragments thereof due to degeneracy of the genetic code and thus encode the same modified CD-NTase polypeptide, or fragment thereof. In another embodiment an isolated nucleic acid molecule of the invention has a nucleotide sequence encoding a protein having an amino acid sequence shown in Table 1, or fragments thereof or a protein having an amino acid sequence which is at least about 70%, 75%, 80%, 85%, 90%, 91%, 92%, 93%, 94%, 95%, 96%, 97%, 98%, 99% or more homologous to an amino acid sequence shown in Table 1, or fragments thereof, or differs by at least 1, 2, 3, 5 or 10 amino acids but not more than 30, 20, 15 amino acids from an amino acid sequence shown in Table 1. In another embodiment a nucleic acid encoding a modified CD-NTase polypeptide consists of nucleic acid sequence encoding a portion of a full-length modified CD-NTase polypeptide of interest that is less than 195, 190, 185, 180, 175, 170, 165, 160, 155, 150, 145, 140, 135, 130, 125, 120 115, 110, 105, 100, 95, 90. 85, 80, 75, or 70 amino acids in length.

It will be appreciated by those skilled in the art that DNA sequence polymorphisms that lead to changes in the amino acid sequences of the modified CD-NTase polypeptides may exist within a population (e.g., a bacterial population). Such genetic polymorphism in the modified CD-NTase genes, for example encoding the amino acid sequences according to SEQ ID NOs. 1-6, may exist among individuals within a population due to natural allelic variation. As used herein, the terms “gene” and “recombinant gene” refer to nucleic acid molecules comprising an open reading frame encoding a modified CD-NTase protein. Such natural allelic variations can typically result in 1-5% variance in the nucleotide sequence of the modified CD-NTase gene. Any and all such nucleotide variations and resulting amino acid polymorphisms in the modified CD-NTase polypeptide that are the result of natural allelic variation and that do not alter the functional activity of the modified CD-NTase polypeptide are intended to be within the scope of the invention. Nucleic acid molecules corresponding to natural allelic variants and homologues of the modified CD-NTase cDNAs encompassed by the present invention can be isolated based on their homology to the modified CD-NTase nucleic acid sequences disclosed herein using the bacterium cDNA, or a portion thereof, as a hybridization probe according to standard hybridization techniques under stringent hybridization conditions (as described herein).

In addition to naturally-occurring allelic variants of the modified CD-NTase polypeptide sequence that may exist in the population, the skilled artisan will further appreciate that changes can be introduced by mutation into the nucleotide sequences shown in Table 1, or fragments thereof, thereby leading to changes in the amino acid sequence of the encoded modified CD-NTase polypeptide, without altering the functional ability of the modified CD-NTase polypeptide. For example, nucleotide substitutions leading to amino acid substitutions of “non-essential” amino acid residues can be made in the sequence shown in Table 1, or fragments thereof. A “non-essential” amino acid residue is a residue that can be altered from the sequence of the modified CD-NTase polypeptide (e.g., the sequence shown in Table 1, or fragments thereof) without significantly altering the activity of the modified CD-NTase polypeptide, whereas an “essential” amino acid residue is required for the modified CD-NTase polypeptide activity. Other amino acid residues, however, (e.g., those that are not conserved or only semi-conserved across bacterial species) may not be essential for activity and thus are likely to be amenable to alteration without altering the modified CD-NTase polypeptide activity.

Accordingly, another aspect encompassed by the present invention pertains to nucleic acid molecules encoding modified CD-NTase polypeptides that contain changes in amino acid residues that are not essential for the modified CD-NTase polypeptide activity. Such modified CD-NTase polypeptides differ in amino acid sequence from an amino acid sequence shown in Table 1, or fragments thereof, yet retain at least one of the modified CD-NTase polypeptide activities described herein. In one embodiment, the isolated nucleic acid molecule comprises a nucleotide sequence encoding a protein, wherein the protein lacks one or more modified CD-NTase polypeptide domains.

An isolated nucleic acid molecule encoding a modified CD-NTase polypeptide homologous to the proteins show in Table 1, or fragments thereof, can be created by introducing one or more nucleotide substitutions, additions or deletions into the nucleotide sequences shown in Table 1, or fragments thereof, or a homologous nucleotide sequence such that one or more amino acid substitutions, additions or deletions are introduced into the encoded protein. Mutations can be introduced into a nucleotide sequence shown in Table 1, or fragments thereof, or to homologous nucleotide sequence by standard techniques, such as site-directed mutagenesis and PCR-mediated mutagenesis. Preferably conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine isoleucine, proline, phenylalanine methionine, tryptophan) branched side chains (e.g., threonine, valine) and aromatic side chains (e.g., tyrosine, phenylalanine, tryptophan, histidine). Thus, a predicted nonessential amino acid residue in the modified CD-NTase polypeptide is preferably replaced with another amino acid residue from the same side chain family.

Alternatively, in another embodiment, mutations can be introduced randomly along all or part of a modified CD-NTase polypeptide coding sequence, such as by saturation mutagenesis, and the resultant mutants can be screened for the modified CD-NTase polypeptide activity described herein to identify mutants that retain the modified CD-NTase polypeptide activity. Following mutagenesis of a nucleotide sequence shown in Table 1, or fragments thereof, the encoded protein can be expressed recombinantly (as described herein) and the activity of the protein can be determined using, for example, assays described herein.

Another aspect of the invention pertains to the use of vectors preferably expression vectors, containing a nucleic acid encoding a modified CD-NTase polypeptide (or a portion thereof), and preferably encoding a sequence according to SEQ ID NOs. 1-6, or fragment thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, where in additional DMA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. In one embodiment, adenoviral vectors comprising a modified CD-NTase nucleic acid molecule are used.

The recombinant expression vectors encompassed by the present invention comprise a nucleic acid of the invention in a form suitable for expression of the nucleic acid in a host cell, which means that the recombinant expression vectors include one or more regulatory sequences, such as a promoter, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in vitro transcription/translation system or in a host cell when the vector is introduced into the host cell). The term “regulatory sequence” is intended to include promoters, enhancers and other expression control dements (e.g., polyadenylation signals).

Another aspect encompassed by the present invention pertains to host cells into which a recombinant expression vector or nucleic acid encompassed by the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences such progeny may not, in fact, be identical to the parent cell, but are still included within the scope of the disclosure as used herein.

A host cell can be any prokaryotic or eukaryotic cell. For example, the modified CD-NTase polypeptide can be expressed in bacterial cells such as E. coli, insect cells, yeast or mammalian cells. Other suitable host cells are known to those skilled in the art. Vector DNA can be introduced into prokaryotic or eukaryotic cells via conventional transformation or transfection techniques. As used herein, the terms transformation” and “transfection” are intended to refer to a variety of art-recognized techniques for introducing foreign nucleic acid (e.g. DNA) into a host cell, including calcium phosphate or calcium chloride co-precipitation DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, NY, 1989), and other laboratory manuals.

A cell culture includes host cells, media and other byproducts. Suitable media for cell culture are well-known in the art. A modified CD-NTase polypeptide or fragment thereof may be secreted and isolated from a mixture of cells and medium containing the polypeptide. Alternatively, a modified CD-NTase polypeptide or fragment thereof may be retained in the cytoplasm and the cells harvested, lysed and the protein or molecular complex isolated. A modified CD-NTase polypeptide or fragment thereof, or synthesis product including preferably 2′3′-cGAMP may be isolated from cell culture medium, host cells, or both using techniques known in the art for purifying proteins or chemical compounds, including ion-exchange chromatography, gel filtration chromatography, ultrafiltration, electrophoresis, and immunoaffinity purification with antibodies specific for particular epitopes of the modified CD-NTase polypeptide, or a fragment thereof, or 2′3′-cGAMP molecule. In some embodiments, the modified CD-NTase polypeptide, or biologically active fragment thereof, and may be fused to a heterologous polypeptide. In certain embodiments, the fused polypeptide has greater half-life and/or cell permeability than the corresponding unfused modified CD-NTase polypeptide, or biologically active fragment thereof. For example, the modified CD-NTase polypeptide may be fused to a cell permeable peptide to facilitate the delivery of the modified CD-NTase polypeptide into the intact cells.

In another embodiment, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well-known in the art and include commercially available kits. Examples of in vitro translation systems include eukaryotic lysates, such as rabbit reticulocyte lysates, rabbit oocyte lysates, human cell lysates, insect cell lysates and wheat ger extracts.

In certain embodiments, the modified CD-NTase polypeptide, or fragment thereof may be synthesized chemically, ribosomally in a cell free system, or ribosomally within a cell. Chemical synthesis may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemo-selective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules

For stable transfection of cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as antibiotics. Nucleic acid encoding a selectable marker can be introduced into a host cell on the same vector as that encoding the modified CD-NTase polypeptide or can be introduced on a separate vector. Cells stably transfected with the introduced nucleic acid can be identified by selection (e.g., cells that have incorporated the selectable marker gene will survive, while the other cells die).

A host cell encompassed by the present invention, such as a prokaryotic or eukaryotic host cell in culture can be used to produce (i.e., express) the CD-NTase polypeptide, and/or 2′3′-cGAMP. Accordingly, the invention further provides methods for producing the modified CD-NTase polypeptide and/or 2′3′-cGAMP using the host cells of the invention. In one embodiment, the method composes culturing the host cell of invention (into which a recombinant expression vector encoding the modified CD-NTase polypeptide has been introduced) in a suitable medium until the CD-NTase polypeptide and/or 2′3′-cGAMP is produced. In another embodiment, the method further comprises isolating the CD-NTase polypeptide and/or 2′3′-cGAMP from the medium or the host cell.

The host cells of the invention can also be used to produce human or non-human transgenic animals and/or cells that, for example, overexpress the modified CD-NTase polypeptide or oversecrete the modified CD-NTase polypeptide. The non-human transgenic animals can be used in screening assays designed to identify agents or compounds, e.g., drugs, pharmaceuticals, etc., winch are capable of ameliorating detrimental symptoms of selected disorders such as cancer, as well as evaluate the use of and/or 2′3′-cGAMP as a therapeutic compound, for example secreted through a probiotic bacteria administered to a subject that is not transgenic or otherwise modified. As used herein, a “transgenic animal” is a non-human animal, preferably a mammal, more preferably a rodent such as a rat or mouse, in which one or more of the cells of the animal includes a transgene. A transgene is exogenous DNA which is integrated into the genome of a cell from which a transgenic animal develops, and which remains in the genome of the mature animal, thereby directing the expression of an encoded gene product in one or more cell types or tissues of the transgenic animal.

The present invention also provides soluble, purified and/or isolated forms of wild-type or modified CD-NTase polypeptides that catalyzes production of the second messenger 2′3′-cGAMP, wherein said polypeptide comprises an amino acid sequence having at least 70% identity to any one of CD-NTase amino acid sequences listed in Table 1 and further comprises a nucleotidyltransferase protein. An amino acid sequence of any modified CD-NTase polypeptide described herein can also be at least 50, 55, 60, 65, 70, 75, 80, 85, 90, 91, 92, 93, 94, 95, 96, 97, 98, 99, or 99.5% identical to a CD-NTase amino acid sequence of any one of CD-NTase amino acid sequences listed in Table 1, or a fragment thereof. In another aspect, the present invention contemplates a composition comprising an isolated wild-type or modified CD-NTase polypeptide described herein and less than about 25%, or alternatively 15%, or alternatively 5%, contaminating biological macromolecules or polypeptides.

The present invention further provides compositions related to producing, detecting, or characterizing a modified CD-NTase polypeptide, or fragment thereof, such as nucleic acids, vectors, host cells and the like. Such compositions may serve as compounds that modulate a modified CD-NTase polypeptide's expression and/or activity, such as antisense nucleic acids.

In certain embodiments, a modified CD-NTase polypeptide of the invention may be a fusion protein containing a domain which increases its solubility and bioavailability and/or facilitates its purification, identification, detection, and/or structural characterization. In some embodiments, it may be useful to express a modified CD-NTase polypeptide in which the fusion partner enhances fusion protein stability in blood plasma and/or enhances systemic bioavailability. Exemplary domains include for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary' domains include domains that alter protein localization in vivo, such as signal peptides, type 2 or 1 secretion system-targeting peptides, transcytosis domains, nuclear localization signals, etc. In various embodiments, a modified CD-NTase polypeptide of the invention may comprise one or more heterologous fusions.

Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope of the invention to include linker sequences between a polypeptide of the invention and the fusion domain in order to facilitate construction of the fusion protein or to optimize protein expression or structural constraints of the fusion protein. In another embodiment, the polypeptide may be constructed so as to contain protease cleavage sues between the fusion polypeptide and polypeptide of the invention in order to remove the tag after protein expression or thereafter. Examples of suitable endoproteases include, for example, Factor Xa and TEV proteases. in some embodiments, the modified CD-NTase polypeptides, or fragments thereof, are fused to an antibody (e.g., IgG1, IgG2, IgG3, IgG4) fragment (e.g., Fc polypeptides).

In still another embodiment, a modified CD-NTase polypeptide may be labeled with a fluorescent label to facilitate their detection, purification, or structural characterization. In an exemplary embodiment, a modified CD-NTase polypeptide of the invention may be fused to a heterologous polypeptide sequence which produces a detectable fluorescent signal, including, for example, green fluorescent protein (GFP), enhanced green fluorescent protein (EGFP), GFPmut2, GFPuv4, enhanced yellow fluorescent protein (EYFP), enhanced cyan fluorescent protein (E-CFP), enhanced blue fluorescent protein (EBFP), citrine and red fluorescent protein from discosoma fdsRED).

In preferred embodiments, the modified CD-NTase polypeptide or portion thereof comprises an amino acid sequence which is sufficiently homologous to an amino acid sequence shown in Table 1 or fragment thereof catalyzes production of 2′3′ cGAMP. The portion of the protein is preferably a biologically active portion as described herein. In another preferred embodiment, the modified CD-NTase polypeptides has an amino acid sequence shown in Table 1, or fragments thereof, or an amino acid sequence which is at least about 50%, 55%, 60%, 65%, 70%, 75%, 80%, 85%, 90%, 91%, 92%, 93%, 94%, 95%, 96%, 97%, 98%, 99% or more homologous to the amino acid sequence shown in Table 1, or fragment thereof.

Biologically active portions of a modified CD-NTase polypeptide include peptides comprising amino acid sequences derived from the amino acid sequence of the modified CD-NTase protein, or the amino acid sequence of a protein homologous to the modified CD-NTase protein which include fewer amino acids than the full-length modified CD-NTase protein or the full-length polypeptide which is homologous to the modified CD-NTase protein, and exhibit at least one activity of the modified CD-NTase protein, namely synthesis of 2′3′-cGAMP. Typically, biologically active portions (peptides, e.g., peptides which are, for example, 50, 55 60, 65 70, 75, 80, 85, 90 95, 100 or more amino acids in length) comprise a domain or motif (e.g., the full-length protein minus the signal peptide). In a preferred embodiment, the biologically active portion of the protein which includes one or more the domains/motifs described herein catalyzes production of 2′3′-cGAMP. Moreover, other biologically active portions, in which other regions of the protein are deleted, can be prepared by recombinant techniques and evaluated for one or more of the activities described herein. Preferably, the biologically active portions of the modified CD-NTase protein include one or more selected domains/motifs or portions thereof having biological activity. In one embodiment, a modified CD-NTase polypeptide fragment of interest consists of a portion of a full-length modified CD-NTase polypeptide that is less than 240, 230, 220, 210, 200, 195. 190, 185, 180, 175, 170, 165, 160, 155, 150. 145, 140, 135, 130, 125, 120, 115, 110, 105, 100, 95, 90, 85, 80, 75, or 70 amino acids in length.

The modified CD-NTase polypeptides of the precent invention can be produced by recombinant DNA techniques. For example, a nucleic acid molecule encoding the protein is cloned into an expression vector (as described above), the expression vector is introduced into a host cell (as described above) and the modified CD-NTase polypeptide is expressed in the host cell, which can produce 2′3′-cGAMP independent of the cGAS pathway, and without the present of a ligand, such as DNA. The modified CD-NTase polypeptide and/or synthesized 2′3′-cGAMP can then be isolated from the cells by an appropriate purification scheme using standard protein purification techniques. In alternative embodiments, the host cell can include, for example a transformed, or genetically modified cell, such as a bacteria that heterologously expresses CD-NTase polypeptide. This host cell can be administered to a subject, such as an animal, and more preferably a human and colonize the subject where the CD-NTase polypeptide synthesizes 2′3′-cGAMP which can be secreted by the host cell and thereby administered to the subject. In a preferred embodiment, the host cell is a probiotic bacterium, and preferably a human enteric probiotic bacterium.

Alternative to recombinant expression, a modified CD-NTase protein, polypeptide, or peptide can be synthesized chemically using standard peptide synthesis techniques. Moreover, modified CD-NTase protein can be isolated from cells (e.g., engineered cells that harboring modified CD-NTase), for example using an anti-CD-NTase antibody. The invention also provides modified CD-NTase chimeric or fusion proteins. As used herein, a modified CD-NTase “chimeric protein” or “fusion protein” comprises a modified CD-NTase polypeptide operatively linked to a non-CD-NTase polypeptide.

A “modified CD-NTase polypeptide” refers to a polypeptide having an amino acid sequence having at least 70% identity to CD-NTase shown in Table 1, whereas a “non-CD-MTase polypeptide” refers to a polypeptide having an amino acid sequence corresponding to a protein which is not substantially homologous to the modified CD-NTase protein, e.g., a protein winch is different from the modified CD-NTase protein and which is derived from the same or a different organism. Within the fusion protein, the term “operatively linked” is intended to indicate that the modified CD-NTase polypeptide and the non-CD-NTase polypeptide are fused in-frame to each other. The non-CD-NTase polypeptide can be fused to the N-terminus or C-terminus of the modified CD-NTase polypeptide. For example, in one embodiment the fusion protein is a modified CD-NTase-GST and/or modified CD-NTase-Fc fusion protein in which the modified CD-NTase sequences, respectively, are fused to the—terminus of the GST or Fc sequences. Such fusion proteins can be made using the modified CD-NTase polypeptides. Such fusion proteins can also facilitate the purification, expression and/or bioavailability of recombinant modified CD-NTase polypeptides. In another embodiment, the fusion protein is a modified CD-NTase protein containing a heterologous signal sequence at its C-terminus. In certain host cells (e.g., mammalian or bacterial host cells), expression and/or secretion of the modified CD-NTase polypeptides and the resulting synthesized 2′3′-cGAMP can be increased through use of a heterologous sequences.

The present invention also pertains to homologues of the modified CD-NTase proteins. Homologues of the modified CD-NTase protein can be generated by mutagenesis, e.g., discrete point mutation or truncation of the modified CD-NTase protein, respectively. As used herein, the term “homologue” refers to a variant form of the modified CD-NTase protein. In one embodiment, treatment of a subject with a homologue having a subset of the biological activities of the naturally occurring form of the protein has fewer side effects in a subject relative to treatment with the naturally occurring form of the modified CD-NTase protein.

In an alternative embodiment, homologues of the modified CD-NTase protein can be identified by screening combinatorial libraries of mutants, e.g., truncation mutants, of the modified CD-NTase protein. In one embodiment, a variegated library of the modified CD-NTase variants is generated by combinatorial mutagenesis at the nucleic acid level and is encoded by a variegated gene library. A variegated library of the modified CD-NTase variants can be produced by, for example, enzymatically ligating a mixture of synthetic oligonucleotides into gene sequences such that a degenerate set of potential modified CD-NTase sequences is expressible as individual polypeptides, or alternatively, as a set of larger fusion proteins (e.g., for phage display) containing the set of the modified CD-NTase sequences therein. There are a variety of methods which can be used to produce libraries of potential modified CD-NTase homologues from a degenerate oligonucleotide sequence. Chemical synthesis of a degenerate gene sequence can be performed in art automatic DNA synthesizer, and the synthetic gene then ligated into an appropriate expression vector. Use of a degenerate set of genes allows for the provision, in one mixture, of all of the sequences encoding the desired se t of potential modified CD-NTase sequences. Methods for synthesizing degenerate oligonucleotides are known in the art (see, e.g., Narang, S. A. (1983) Tetrahedron 39:3; Itakura et al. (1984) Amu. Rev. Biochem. 53:323; Itakura et al. (1984} Science 198:1056; Ike et al. (1983) Nucleic Acid Res. 11:477

In addition, libraries of fragments of the modified CD-NTase protein coding can be used to generate a population of the modified CD-NTase fragments for screening and subsequent selection of homologues of a modified CD-NTase protein. In one embodiment, a library of coding sequence fragments can be generated by treating a double stranded PCR fragment of a modified CD-NTase coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal and internal fragments of various sizes of the modified CD-NTase protein.

The modified CD-NTase nucleic acid and polypeptide molecules described herein may be used to produce 2′3′-cGAMP. For example, the modified CD-NTase nucleic acid or polypeptide molecules may be delivered into a cell, or an organism cultured at an optimal condition so that the modified CD-NTase nucleic acid or polypeptide molecules catalyze 2′3′-cGAMP synthesis. The delivery method is known in the art and also described herein. For example, the modified CD-NTase nucleic acid or polypeptide molecules may be delivered using chemical vehicles like liposomes or through viral delivery. In other embodiments, the modified CD-NTase nucleic acid or polypeptide molecules may be contacted with nucleotide substrates in a cell-fee condition where buffers, ions, and/or ligands required for the catalytic activity of the modified CD-NTase are supplied.

2′3′-cGAMP synthesis by the CD-NTases of the invention can be modulated further in addition to expressing the CD-NTases. For example, the nucleotide substrates may be modified, or unnatural nucleotides as described in the definitions, so that the 2′3′-cGAMP synthesized may include modified or unnatural nucleotides. Methods for identifying, purifying, and/or characterizing the produced 2′3′-cGAMP are known in the art. The CD-NTases themselves and/or 2′3′-cGAMP produced using the modified CD-NTase nucleic acid and polypeptide molecules described herein, can be used as therapeutics, preferably as part of pharmaceutical compositions as described herein, for example through probiotic delivery systems.

The nucleic acid molecules of the invention can be inserted into vectors and used as gene therapy vectors. Gene therapy vectors can be delivered to a subject by, for example, intravenous injection local administration (see U.S. Pat. No. 5,328,470) or by stereotactic injection (see e.g., Chen et at (1994) Proc. Natl. Acad. Set USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells winch produce the gene delivery system.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

The term “STING pathway” or “cGAS-STING pathway” refers to a STING-regulated innate immune pathway, which mediates cytosolic DMA-induced signaling events. Cytosolic DNA binds to and activates cGAS, which catalyzes the synthesis of 2′3′-cGAMP from ATP and GTP. 2′3′-cGAMP binds to the ER adaptor STING, which traffics to the ER-Golgi intermediate compartment (ERG1C) and the Golgi apparatus. STING then activates IKK and TBK1. TBK1 phosphorylates STING, which in turn recruits IRF3 for phosphorylation by TBK1. Phosphorylated IRF3 dimerizes and then enters the nucleus, where it functions with NF-κB to turn on the expression of type I interferons and other immunomodulatory molecules. The cGAS-STING pathway not only mediates protective immune defense against infection by a large variety of DNA-containing pathogens but also detects tumor-derived DNA and generates intrinsic antitumor immunity. However, aberrant activation of the CGAS-STING pathway by self-DNA can also lead to autoimmune and inflammatory' disease.

The term “cGAS” or “Cyclic GMP-AMP Synthase”, refers to nucleotidyltransferase that catalyzes the formation of cyclic GMP-AMP (cGAMP) from ATP and GTP

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens. As noted above, the terms protein and peptide also include protein fragments, epitopes, catalytic sites, signaling sites, localization sites and the like. A peptide or protein may further be a fusion or chimera peptide, which a used herein means a peptide having at least a first and second domain or moiety. As described herein, in certain embodiment various peptides, including chimeric peptides or oligonucleotides, such as RNA molecules may be co-expressed. In some embodiments the elements may be co-expressed from a single expression vector having one or more expression cassettes, or from separate expression vectors having one or more expression cassettes. Such expression may also be the result of transient or stable transformation of a cell. As used herein, a “functional” polypeptide or “functional fragment” is one that substantially retains at least one biological activity normally associated with that polypeptide (e.g., nucleosome formation).

The terms “polypeptides” and “proteins” are, where applicable, used interchangeably herein. They may be chemically modified, e.g. post-translationally modified. For example, they may be glycosylated or comprise modified amino acid residues. They may also be modified by the addition of a signal sequence to promote their secretion from a cell where the polypeptide does not naturally contain such a sequence. They may be tagged with a tag. They may be tagged with different labels which may assist in identification of the proteins in a molecular complex. Polypeptide s/proteins for use in the Invention may be in a substantially isolated form. It will be understood that the polypeptide/protein may be mixed with earners or diluents which will not interfere with the intended purpose of the polypeptide and still be regarded as substantially isolated.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art and is understood as included in embodiments where it would be appropriate. Nucleotides may be referred to by their commonly accepted single-letter codes. Unless otherwise indicated, amino acid sequences are written left to right in amino to carboxyl orientation, respectively. Amino acids may be referred to herein by either their commonly known three letter symbols or by the one-letter symbols as generally understood by those skilled in the relevant art.

The term “fragment,” as applied to a polynucleotide, can further be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., at least 90%, 92%, 95%, 98%, 99% identical) to the 25 reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence 30 according to the invention. In some embodiments, such fragments can comprise, consist essentially of, and/or consist of oligonucleotides having a length of less than about 8, 10, 12, 15, 20, 25, 30, 35, 40, 45, 50, 75, 100, 150, or 200 consecutive nucleotides of a nucleic acid or nucleotide sequence according to the invention.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, a 10 isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least 85% pure, and more preferably at least 95% pure, and most preferably at least 99% pure.

An “isolated protein” refers to a protein that is substantially free of other proteins, cellular material, separation medium and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a polypeptide or fragment thereof, in which the protein is separated from cellular components of the cells from which it is isolated or recombinantly produced hi one embodiment, the language “substantially free of cellular material” includes preparations of a modified CD-NTase polypeptide or fragment thereof having less than about 30% (by dry-weight) of non-CD-NTase protein (also referred to herein as a “contaminating protein”), more preferably less than about 20% of non-CD-NTase protein, still more preferably less than about 10% of non-CD-NTase protein, and most preferably less than about 5% non-CD-NTase protein. When antibody polypeptide, peptide or fusion protein or fragment thereof e.g., a biologically active fragment thereof, is recombinantly produced, it is also preferably substantially free of culture medium, i.e., culture medium represents less than about 20%, and more preferably less than about 10%, and most preferably less than about 5% of the volume of the protein preparation

The term “modified CD-NTase polypeptide” refers to CD-NTase polypeptide that is different from that found in nature, in its native or original cellular or body environment. The term “modification” as used herein refers to all modifications of a protein, DNA, or protein-DNA complex of the invention including cleavage and addition or removal of a group. The “modified CD-NTase polypeptide” of this invention may be, e.g. homolog, derivative, or fragment of native CD-NTase polypeptide having an amino acid sequence listed in Table 1. Preferably, the “modified CD-NTase polypeptide” has one or more following biological activities: a) circular or linear nucleotide-based second messenger synthesis; b) active enzyme conformation; and c) STING or RECON pathway regulation. The term “modified CD-NTase nucleic acid” refers to nucleic acid (e.g., DNA, mRNA) that encodes the modified CD-NTase polypeptide of described herein. As used herein, the term “nucleotide-based second messenger” refers to a second messenger having a relatively small number (e.g., one, two, or three) of nucleotides or derivatives thereof that transduces signals originating from changes in the environment or in intracellular conditions into appropriate cellular responses. It can be circular or linear. In one embodiment, the nucleotide-based second messenger is a cyclic dinucleotide which includes but is not limited to 2′3′=cGAMP. the nucleotide-based second messenger may contain modified or unnatural nucleotides. The modified nucleotides can be naturally occurring modified RNA base analogs (Limbach et al. (1994) Nucleic Acids Res 22:2183-2196: Cantara et al. (2011) Nucleic Adds Res 39:0195-0201; Czerwoniec et al. (2009) Nucleic Acids Res 37: D118-D121; Grosjean et al. (1998) Modification and Editing of RNA. ASM Press, Washington DC.)

As used herein, an “agonist” means a substance, having the function of binding/activating to a receptor or to produce a biological response.

A “marker”, or “tag” as used herein, refers to a molecule that can be used for identification, detection, purification, or isolation. In an embodiment, the marker comprises a small molecule, a peptide, a polypeptide, or a labeled amino acid or nucleotide. In an embodiment, the marker generates a signal for detection, e.g., a radioactive signal, a chemiluminescent signal, a fluorescent signal, or a chromogenic signal. For example, the marker is a dye, a fluorophore, a reporter enzyme (e.g., a photoprotein, luciferase), a fluorescent peptide, or a radionuclide. The generated signal can be detected by a variety of assays known in the art, such as fluorescence microscopy, fluorescence-activated cell sorting, gel electrophoresis, and spectrophotometry.

A “subject” is any organism of interest, generally a mammalian subject, and preferably a human subject.

The term “probiotic” refers to a microorganism, such as bacteria, which may colonize a host. A probiotic may include endosymbiotic bacteria, or naturally occurring flora that may permanently to temporarily colonize an animal, such as a human. Probiotic organisms may also include human gut probiotic strains.

The term “expression,” as used herein, or “expression” of a coding sequence (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons).

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

The term “sequence identity” or “identity,” as used herein in the context of two nucleic acid or polypeptide sequences, refers to the residues in the two sequences that are the same when aligned for maximum correspondence over a specified comparison window. As used herein, the term “percentage of sequence identity” may refer to the value determined by comparing two optimally aligned sequences (e.g., nucleic acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

Notably, disclosure of a nucleotide sequence also specifically includes with the disclosure its corresponding RNA and amino acid sequence, and vice versa.

Polynucleotide sequences may have substantial identity, substantial homology, or substantial complementarity to the selected region of the target gene. As used herein “substantial identity” and “substantial homology” indicate sequences that have sequence identity or homology to each other. Generally, sequences that are substantially identical or substantially homologous will have about 75%, 80%, 85%, 90%, 91%, 92%, 93%, 94%, 95%, 96%, 97%, 98%, 99%, or 100% sequence identity wherein the percent sequence identity is based on the entire sequence and is determined by GAP alignment using default parameters (GCG, GAP version 10, Accelrys, San Diego, CA). GAP uses the algorithm of Needleman and Wunsch ((1970) J Mol Biol 48:443-453) to find the alignment of two complete sequences that maximizes the number of matches and minimizes the number of sequence gaps. Sequences which have 100% identity are identical. “Substantial complementarity” refers to sequences that are complementary to each other, and are able to base pair with each other. In describing complementary sequences, if all the nucleotides in the first sequence will base pair to the second sequence, these sequences are fully complementary.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10. The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default BLOSUM62 matrix set to default parameters. Nucleic acid sequences with even greater similarity to the reference sequences will show increasing percentage identity when assessed by this method.

As used herein, the term “homologous” with regard to a contiguous nucleic acid sequence, refers to contiguous nucleotide sequences that hybridize under appropriate conditions to the reference nucleic acid sequence. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

Homologs, variants and alleles of the target molecules or proteins of the invention can be identified by conventional techniques. As used herein, a homolog or variant to a polypeptide is a polypeptide from a source that has a high degree of structural similarity to the identified polypeptide. The terms “derivatives”, “analogs” or “variants” as used herein include, but are not limited, to molecules comprising regions that are substantially homologous to the modified CD-NTase polypeptide, in various embodiments, by at least 30%, 40%, 50%, 60%, 70%, 80%, 90%, 95% or 99% identity over an amino acid sequence of identical size or when compared to an aligned sequence in which the alignment is done by a computer homology program known in the art, or whose encoding nucleic acid is capable of hybridizing to a sequence encoding the component protein under stringent, moderately stringent, or non-stringent conditions. It means a protein which is the outcome of a modification of the naturally occurring protein, by amino acid substitution, deletions and additions, respectively, which derivatives still exhibit the biological function of the naturally occurring protein although not necessarily to the same degree. The biological function of such proteins can e.g., be examined by suitable available in vitro assays as provided in the invention.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

As used herein, the term “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into such a bacteria.

As used herein, the term “host cell” is intended to refer to a cell into which a nucleic-acid encompassed by the present invention, such as a recombinant expression vector encompassed by the present invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not. in fact, be identical to the parent cell, but are still included within the scope of the term as used herein.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are several types of vectors including virus, plasmid, bacteriophages, cosmids, and bacteria.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector is polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. As provided below, the table contains information about which nucleic acid codons encode which amino acids.

Amino acid Nucleic acid codons

| | Nucleic Acid |
|---|---|
| Amino Acid | Codons |
| Ala/A | GCT, GCC, GCA, |
| | GCG |
| Arg/R | CGT, CGC, CGA, |
| | CGG,AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, |
| | GGG |
| His/H | CAT, CAC |
| Ile/I | ATT, ATC, ATA |
| Leu/L | TTA, TTG, CTT, |
| | CTC, CTA, CTG |
| Lys/K | AAA, AAG |
| Met/M | ATG |
| Phe/F | TTT, TTC |
| Pro/P | CCT, CCC, CCA, |
| | CCG |
| Ser/S | TCT, TCC, TCA, |
| | TCG, AGT, AGC |
| Thr/T | ACT, ACC, ACA, |
| | ACG |
| Trp/W | TGG |
| Tyr/Y | TAT, TAC |
| Val/V | GTT, GTC, GTA, |
| | GTG |

In addition to the degenerate nature of the nucleotide codons which encode amino acids, alterations in a polynucleotide that result in the production of a chemically equivalent amino acid at a given site, but do not affect the functional properties of the encoded polypeptide, are well known in the art. “Conservative amino acid substitutions” are those substitutions that are predicted to interfere least with the properties of the reference polypeptide. In other words, conservative amino acid substitutions substantially conserve the structure and the function of the reference protein. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine or histidine, can also be expected to produce a functionally equivalent protein or polypeptide. As provided below, the table provides a list of exemplary conservative amino acid substitutions. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

Amino Acids and Conservative Substitutes

| | Conservative |
|---|---|
| Amino Acid | Substitute |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, |
| | Glu |
| Ile | Leu, Val |
| Leu | Ile, Val |
| Lys | Arg, Gln, Glu |
| Met | Ile, Leu |
| Phe | His, Leu, Met, |
| | Trp, Tyr |
| Ser | Cys, Thr |
| Thr | Ser, Val |
| Trp | Phe, Tyr |
| Tyr | His, Phe, Trp |
| Val | Ile, Leu, Thr |

Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983). Additional methods are known by those of ordinary skill in the art.

As used herein, the term “endogenous” refers to any material from or produced inside an organism, cell, tissue or system.

As used herein, the term “exogenous” or “heterologous” refers to any material introduced from or produced outside an organism, cell, tissue or system.

The term “recombinant” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed—over-expressed, under expressed or not expressed at all.

The terms “transgenic,” “transformed,” “transformation,” and “transfection” are similar in meaning to “recombinant.” “Transformation,” “transgenic,” and “transfection” refer to the transfer of a polynucleotide into the genome of a host organism or into a cell. Such a transfer of polynucleotides can result in genetically stable inheritance of the polynucleotides or in the polynucleotides remaining extra-chromosomally (not integrated into the chromosome of the cell). Genetically stable inheritance may potentially require the transgenic organism or cell to be subjected for a period of time to one or more conditions which require the transcription of some or all of the transferred polynucleotide in order for the transgenic organism or cell to live and/or grow. Polynucleotides that are transformed into a cell but are not integrated into the host's chromosome remain as an expression vector within the cell. One may need to grow the cell under certain growth or environmental conditions in order for the expression vector to remain in the cell or the cell's progeny. Further, for expression to occur the organism or cell may need to be kept under certain conditions. Host organisms or cells containing the recombinant polynucleotide can be referred to as “transgenic” or “transformed” organisms or cells or simply as “transformants,” as well as recombinant organisms or cells.

A genetically altered organism is any organism with any change to its genetic material, whether in the nucleus or cytoplasm (organelle). As such, a genetically altered organism can be a recombinant or transformed organism. A genetically altered organism can also be an organism that was subjected to one or more mutagens or the progeny of an organism that was subjected to one or more mutagens and has changes in its DNA caused by the one or more mutagens, as compared to the wild-type organism (i.e., organism not subjected to the mutagens). Also, an organism that has been bred to incorporate a mutation into its genetic material is a genetically altered organism.

The terms “approximately” and “about” refer to a quantity, level, value or amount that varies by as much as 30%, or in another embodiment by as much as 20%, and in a third embodiment by as much as 10% to a reference quantity, level, value or amount. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gaminopathy, and melanomas, breast cancer, lung cancer bronchus cancer. colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary' tract cancer, small bowel or appendix cancer, salivary gland cancer, thyroid gland cancer, adrenal gland cancer, osteosarcoma, chondrosarcoma, and the like.

A “pharmaceutical composition” or “pharmaceutical composition of the invention” refers to a 2′3′-cGAMP, or a peptide according to Table 1, sometimes referred to as a compound of the invention or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients. In other embodiments, the pharmaceutical composition further comprises at least one additional antibiotic, such as through a co-treatment. As used herein, a “pharmaceutically acceptable carrier” refers to a carrier or diluent that does not cause significant irritation to an organism and does not abrogate the biological activity and properties of the administered composition of the invention. The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form.

Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin, and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages. Exemplary parenteral administration forms include solutions or suspensions of active compounds in sterile aqueous solutions, for example, aqueous propylene glycol or dextrose solutions. Such dosage forms may be suitably buffered, if desired.

A pharmaceutical composition of the invention may be administered as single agents, for example a pharmaceutical composition of a compound of the invention, or a pharmaceutical composition of a compound of the invention, or may be administered in combination with an antibiotic, and preferably polymyxin family of antibiotic, such as polymyxin A or colistin. In some embodiments, the methods provided result in one or more of the following effects: (1) treating a MDR gram-negative bacterial infection in a subject, and preferably a polymyxin resistant Gram-negative bacteria; (2) inhibiting growth of gram-negative bacteria; (3) preventing infection of a MDR gram-negative bacterial infection in a subject; and (4) sensitizing or re-sensitizing a MDR gram-negative bacterial infection to an antibiotic and preferably polymyxin family of antibiotic, such as polymyxin A or colistin. Pharmaceutical compositions suitable for the delivery of one or more compounds of the invention as described herein, and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least 0.1% of active compound. The percentage of the compositions and preparation can, of course, be varied and can conveniently be between about 1 to about 10% of the weight of the unit. The amount of active compound in such therapeutically useful compositions is such that a suitable dosage will be obtained. Typical compositions or preparations according to the invention are prepared such that an oral dosage unit form contains from about 1 to about 1000 mg of active compound.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound can be incorporated into sustained-release preparations and formulation. In addition to the common dosage forms set out above, the compounds of the invention may also be administered by controlled release means and/or delivery devices capable of releasing the active ingredient (prenylation inhibitor) at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. Examples of controlled release pharmaceutical compositions and delivery devices that may be adapted for the administration of the active ingredients of the present invention are described in U.S. Pat. Nos. 3,847,770; 3,916,899; 3,536,809; 3,598,123; 3,630,200; 4,008,719; 4,687,610; 4,769,027; 5,674,533; 5,059,595; 5,591,767; 5,120,548; 5,073,543; 5,639,476; 5,354,566; and 5,733,566, the disclosures of which are hereby incorporated by reference.

Pharmaceutical compositions for use in the methods of the present invention may be prepared by any of the methods of pharmacy, but all methods include the step of bringing the active ingredient into association with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent.

The compound of the invention can also be administered parenterally. Solutions of the active compound as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the compound of the invention in the required amount in the appropriate solvent with various other ingredients enumerated above, as required, followed by filtered sterilization. Generally, dispersions are prepared by incorporating the various sterilized ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The compounds of the invention can be administered to a mammal alone or in combination with pharmaceutically acceptable carriers, as noted above, the proportion of which is determined by the solubility and chemical nature of the compound, chosen route of administration and standard pharmaceutical practice.

The physician can readily determine the dosage of the present therapeutic agents which will be most suitable for prophylaxis or treatment, and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. “Treating” or “treatment” of a disease includes: (1) preventing the disease, i.e., causing the clinical symptoms of a bacterial infection not to develop in a mammal that may be exposed to or predisposed to the infection but does not yet experience or display symptoms of the infection; (2) inhibiting the disease, i.e., arresting or reducing the development of the infection or its clinical symptoms; or (3) relieving the disease, i.e., causing regression of the infection or its clinical symptoms.

When describing a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product. As used herein, the terms “those defined above” and “those defined herein” when referring to a variable incorporates by reference the broad definition of the variable as well as any narrow definitions, if any.

As defined herein, with respect to any peptide or genetic sequence the terms “derived from” or “from” means directly isolated or obtained from a particular source or alternatively having identifying characteristics of a substance or organism isolated or obtained from a particular source. In the event that the “source” is an organism, “derived from” or “from” means that it may be isolated or obtained from the organism itself or from the medium used to culture or grow said organism.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “any combination thereof” as used herein refers to all permutations and combinations of the listed items preceding the term. For example, “A, B, C, or any combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The disclosure now being generally described will be more readily understood by reference to the following examples, which are included merely for the purposes of illustration of certain aspects of the embodiments of the present disclosure. The examples are not intended to limit the disclosure, as one of skill in the art would recognize from the above teachings and the following examples that other techniques and methods can satisfy the claims and can be employed without departing from the scope of the claimed disclosure.

### EXAMPLES

### Example 1: Overview and Experimental Results

Cyclic oligonucleotides are signaling molecules that play essential roles in cellular physiology and immune signaling throughout the tree of life. In humans, cyclic GMP-AMP synthase (cGAS) produces the cyclic dinucleotide 2′,3′-cyclic GMP-AMP (2′,3′-cGAMP) in response to binding dsDNA in the cytosol, which is often found during infection, cellular stress, and cancer (1). 2′,3′-cGAMP binds to the transmembrane receptor Stimulator of Interferon Genes (STING), which activates downstream inflammatory pathways, antiviral signaling, and programmed cell death (2-4). In this way, cGAS acts as sensor and STING acts as an adaptor to transmit the signaling cascade in response to binding cyclic dinucleotides. STING can also be activated via intercellular transport of 2′,3′-cGAMP (5-10), through viral packaging (11), and by bacterially derived cyclic dinucleotides (12-14), allowing rapid amplification of the innate immune response during cellular stress. Consequently, the cGAS-STING pathway is implicated in several disease pathologies including infection, cancer, and autoimmunity and is a target of host-directed immunotherapy (1-4)

Bacteria encode signaling pathways related to metazoan cGAS-STING called cyclic oligonucleotide-based antiphage signaling systems (CBASS) that restrict bacteriophage replication as a part of the bacterial innate immune system (15-19). CBASS operons encode structural homologs of cGAS called cGAS/DncV-like nucleotidyltransferase (CD-NTases) that are activated during phage infection and produce cyclic oligonucleotides within infected cells to initiate the immune response (15, 20-23). The cyclic oligonucleotide binds to a cognate intracellular CD-NTase-associated protein (Cap), which activates its effector function, leading to restriction of viral replication through diverse mechanisms such as growth arrest or cell death (15, 18, 19, 22, 24). CBASS pathways signal via a range of cyclic oligonucleotides unique to bacteria, including cyclic trinucleotides, and isoforms of cGAMP such as 3′,3′-cGAMP and 3′,2′-cGAMP (15, 21, 25-27). However, the 2′,3′-cGAMP isoform has not been identified in bacteria. Motivated by the critical role of 2′,3′-cGAMP in human cGAS-STING signaling, Applicants sought to investigate if bacteria also encode 2′,3′-cGAMP signaling pathways.

In this Application Applicants discovered a 2′,3′-cGAMP signaling system in bacteria that restricts phage replication by triggering premature cell lysis via a Cap14 effector. These findings expand the role of 2′,3′-cGAMP-mediated immune signaling beyond metazoan cGAS-STING and extend it to bacterium-phage conflict. There are multiple correlations between microbiome constituents and human health and disease (53-56). An intriguing possibility is that bacteria serve as a reservoir of 2′,3′-cGAMP which may antagonize or synergize with human STING-mediated signaling in cancer, infection, or autoimmunity. It is plausible that bacterial 2′,3′-cGAMP could be released into the extracellular space during CBASS-mediated bacteriolysis. Extracellular 2′,3′-cGAMP is imported by mammalian cells and activates STING in paracrine leading to amplified immune responses (5-8, 57). STING binds 2′,3′-cGAMP with high affinity (K_{D} of ˜4 nM) (58), therefore, even minute amounts of exogenously produced 2′,3′-cGAMP could have a significant impact in shifting the balance of STING-mediated signaling which may be beneficial or detrimental to the host depending on cellular context. It will be of interest to identify the activation factors of these systems and phage specificity within the native bacterial populations.

Our investigation of the Cap14 effector reveals its function as a membrane-perforating receptor triggered by cyclic oligonucleotide binding. The Cap14 effector was hypothesized to function as a pore-forming domain when it was first reported through computational discovery (18). Subsequent studies observed membrane disruption for the CBASS transmembrane effectors Cap14, Cap15 (S2™-β), and Cap16 (2™-NUDIX) using an induced expression system in E. coli but direct evidence for pore-formation was not explored (33, 59). Our electrophysiological analysis of full-length BtCap14 in artificial lipid bilayers indicates that the protein directly forms cGAMP-dependent channels in the absence of other cellular components, thus demonstrating a direct and measurable effector function. This is supported by our observations of gross membrane disruption, membrane blebbing, and cell lysis during the biological context of phage infection, which resembles features of eukaryotic programmed cell death. The selectivity of BtCap14 for Cl⁻ was unexpected and distinguishes Cap14 from other pore-forming proteins which often form large nonselective pores. Cl⁻ efflux resulted in membrane depolarization. In the case of BtCap14, this rapidly progressed to membrane deformation and cell lysis, possibly through osmotic destabilization. Cap14 effectors are found in bacteria that inhabit diverse niches; thus, it is likely that they exhibit ion specificity, ligand selectivity, and additional regulatory mechanisms that are adapted for those environments. Saf-2™-SAVED effectors are also encoded within CRISPR operons, suggesting additional roles in antiviral immunity beyond CBASS (18).

The filamentous assembly that Applicants observed for cGAMP-bound BtCap14 is consistent with a growing theme of supramolecular assembly in bacterial immune components (60). Cyclic oligonucleotide binding to SAVED and STING domains initiate oligomerization which is required for activity of fused effector domains such as TIR NADases and CHAT proteases by rearranging the effector domains to build a composite active site contributed by individual monomers (17, 27, 38, 40, 51). Applicants propose that for Cap14, cGAMP-induced oligomerization of the SAVED domain initiates structural rearrangements of the Saf-2™ domain in the membrane to form the ion-conducting pore. The mechanistic basis for this pore remains enigmatic as Applicants were unable to resolve the Saf-2™ domain in our CryoEM reconstruction. It is plausible that multimerization allows assembly of multiple small pores within the filament, based on the amount of cGAMP present, perhaps allowing for controlled regulation of ion flux which may have additional unexplored roles in different infection conditions or biological contexts. Importantly Applicants did observe heterogeneous oligomer assemblies even in the absence of cGAMP; which is also a feature of other CDN receptors, and human STING (33, 60).

Regardless, our data indicates that the channel activity and antiphage protective capability of Cap14 is strictly dependent on cGAMP. Applicants propose that cGAMP binding likely triggers an irreversible multimerization leading to cell death. The structure and mechanism of Cap14 is distinct from other membrane depolarizing/disrupting immune effectors such as Cam1(61), Csx28 (62), gasdermins (63-65), which form structures with a defined pore-like architecture. In contrast, the mechanism of Cap14 is most similar to human STING which also forms dimers, and multimerizes upon binding cGAMP; albeit by a different binding mechanism (46, 47). Recent reports of human STING functioning as a proton channel (44, 45), coupled with our observation that the bacterial STING homologue, Cap13, also mediates cell lysis, suggests that membrane disruption is a conserved feature of TM-STING receptors from bacteria to eukaryotes. Applicants propose a model in which Cap14, and Cap13 effectors bind cyclic dinucleotides produced by CD-NTases during phage infection, form oligomeric assemblies that mediate depolarization through selective ion flux, and execute cell lysis to prevent viral replication (FIG. 7). In the case of human STING, the proton channel activity is key for activation of non-interferon mediated pathways (44, 45). It will be interesting to determine how (and if) this mechanism evolved from its bacterial counterparts, and to investigate ion flux as a primordial feature of immune regulation.

Our engineered biosensor: Chimera 10 provides proof-of-principle for the use of bacterial cGAMP receptors as a platform to develop new CDN-activated biotechnology. Detection of 2′,3′-cGAMP was previously limited to STING-based sensors (68), RNA-based probes (69), mass-spectrometry, and NMR (70). An advantage of Chimera 10 is exquisite selectivity for 2′,3′-cGAMP over other isomers and alternative cyclic dinucleotides, its ease of recombinant production, and its applicability to high-throughput screening compared to existing methods. Applicants envision that Chimera 10 will be applied to detect 2′,3′-cGAMP in contexts ranging from accelerated discovery of new cGAMP synthases, detection of 2′,3′-cGAMP production in bacterial physiology, and in mammalian cells to monitor cGAS activation in disease models and for therapeutic development. This biosensor also highlights the plasticity of a SAVED domain to activate two vastly different effector domains. This quality of SAVED is likely key to the evolution of CBASS systems through generating effector and nucleotide second messenger diversity, which may be an evolutionary advantage for bacteria in their conflict with phage and ecological niche (18, 50). Harnessing this modularity will allow for the development of programmable CDN-activated sensors enabling precise discovery and study of these molecules in biological processes across the tree of life.

### Example 2: Discovery of Bacterial CD-NTases that Synthesize 2′,3′-cGAMP

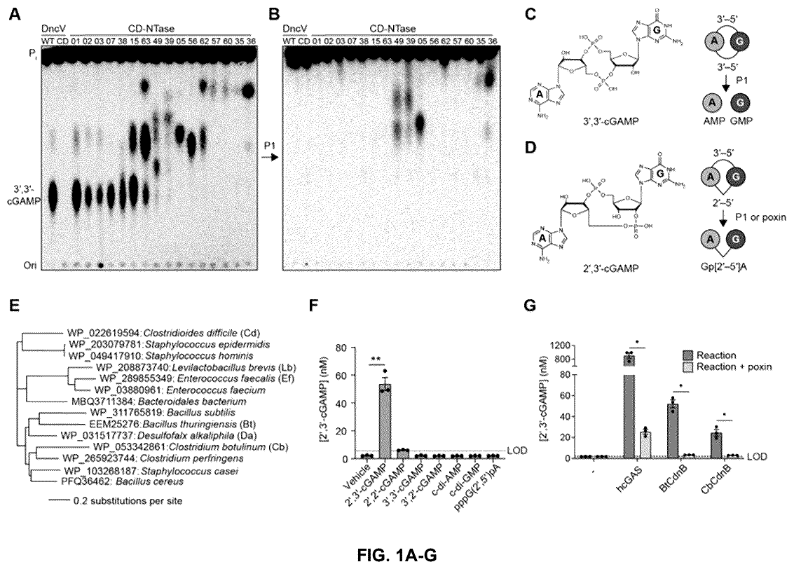

To identify bacterial CD-NTases that synthesize 2′,3′-cGAMP, Applicants screened the enzymatic products produced by a panel of CD-NTases which Applicants identified previously (20). Recombinant CD-NTases were incubated with α³²P-radiolabelled NTPs to monitor cyclic dinucleotide production. The reaction was quenched by treatment with calf intestinal phosphatase, and the reaction products were resolved using thin layer chromatography (TLC) (FIG. 1A). To specifically identify 2′-5′ bonds in CD-NTase reaction products, the reactions were further digested with PI endonuclease, an enzyme that exclusively hydrolyzes 3′-5′ phosphodiester linkages (FIG. 1B). Cyclic oligonucleotides composed of only 3′-5′ phosphodiester linkages, such as 3′,3′-cGAMP, are degraded with PI treatment (FIG. 1C). In contrast, cyclic oligonucleotides composed of one 2′-5′ and one 3′-5′ phosphodiester linkage such as 2′,3′-cGAMP, only the 3′-5′ linkage is hydrolyzed by PI and the remaining 2′-5′-linked linear oligonucleotide is preserved (FIG. 1D). Using this approach, Applicants identified five CD-NTases (49, 39, 05, 35, and 36) that produced P1-resistant ³²P-radiolabelled products (FIG. 1B).

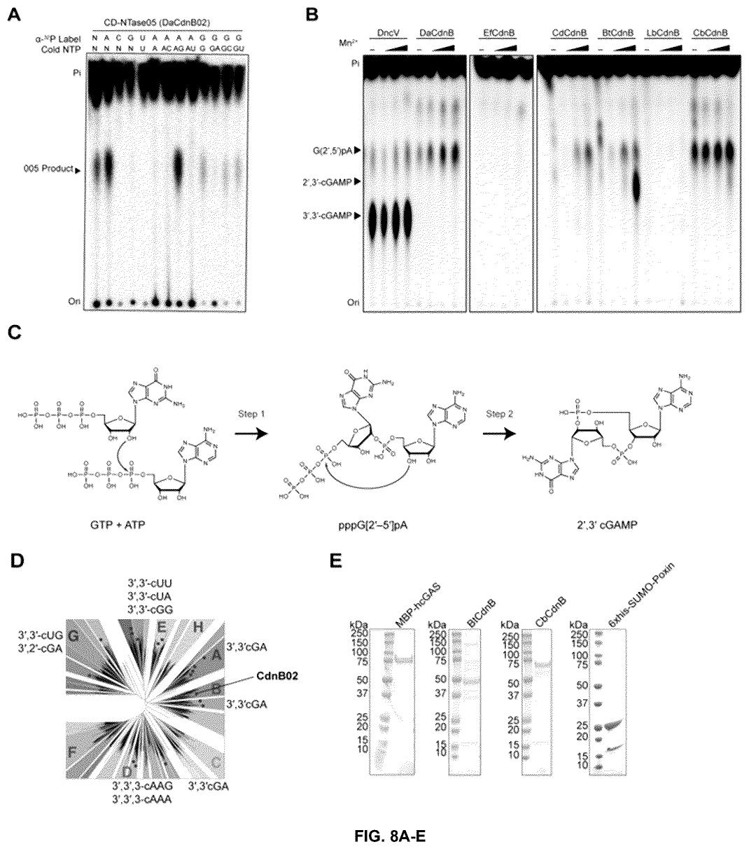

Applicants selected CD-NTase05 from Desulfofalx alkaliphila for further analysis and found that this enzyme only produced a product when ATP and GTP were provided as substrates and α³²P-radiolabel was only protected from phosphatase when [α³²P] ATP was used (FIG. 8A). These findings suggested that CD-NTase05 produced the linear nucleotide pppG(2′,5′) pA in the reaction conditions used. CD-NTases and cGAS produce cyclic dinucleotides by first catalyzing synthesis of a linear dinucleotide intermediate from two NTPs, followed by catalyzing a second reaction that closes the linear intermediate into a cyclic dinucleotide (FIG. 8C) (20). Mammalian cGAS produces linear pppG(2′,5′) pA as an intermediate (28). Our results suggested that CD-NTase05 likely catalyzed the same reaction, however, the reaction may be incomplete due to buffer conditions or lack of unknown activation factors.

Applicants searched for CD-NTase05 homologs to identify additional enzymes that may synthesize 2′,3′-cGAMP. Previous analysis of CD-NTases organized these enzymes into sequence-related clades denoted A-H and showed that enzymes within clades produced similar cyclic oligonucleotides (20). CD-NTase05 is a member of Clade B02 (FIG. 8C) and Applicants renamed this enzyme DaCdnB (D). alkaphilum cGAS DncV-like nucleotidyltransferase from clade B). Applicants identified homologues of DaCdnB and selected a subset for characterization (FIG. 1E, FIG. 8B). Homologues from Bacillus thuringiensis (BtCdnB) and Clostridium botulinum (CbCdnB) synthesized oligonucleotide products that migrated similarly to DaCdnB. A consistent issue with evaluating CD-NTases is that Applicants do not know their cognate activator provided by phage during infection. In some cases this can be overcome by supplementing the reaction with manganese (Mn²), which activates nucleotidyltransferase such as cGAS and CD-NTases (20, 29, 30). Under these conditions, BtCdnB and CbCdnB produced a reaction product with a migration pattern resembling 2′,3′-cGAMP and Applicants hypothesized that Mn² completes the CdnB reaction from linear pppG(2′,5′) pA to 2′,3′-cGAMP (FIG. 8B).

Applicants confirmed that the final reaction product of BtCdnB and CbCdnB was indeed 2′,3′-cGAMP by subjecting the reaction products to an ELISA and measuring nucleotide only when enzyme was added (FIG. 1G). The signal for 2′,3′-cGAMP was highly specific and Applicants did not detect cross-reactivity for the other isoforms of cGAMP, linear pppG(2′,5′) pA, cyclic di-AMP, or cyclic di-GMP (FIG. 1F). Applicants further confirmed the reaction products were indeed 2′,3′-cGAMP by treating the reaction products with VacV Poxin, a 2′,3′-cGAMP-specific hydrolase (31, 32), which significantly reduced the detectable signal (FIG. 1G). Collectively these data indicate that the BtCdnB and CbCdnB enzymes synthesize 2′,3′-cGAMP.

### Example 3: 2′,3′-cGAMP Signaling Restricts Phage Replication by Initiating Bacterial Cell Lysis

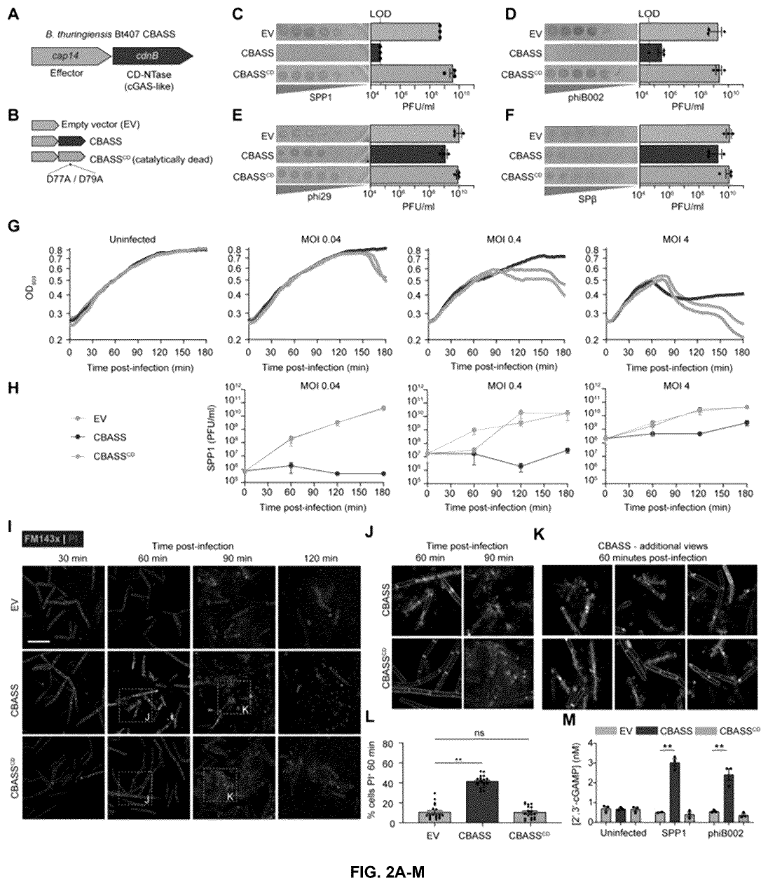

BtCdnB is encoded in a type I CBASS system, a two-gene system from B. thuringiensis Bt407 (hereafter referred to as BtCBASS) which consists of the CD-NTase and a predicted transmembrane effector named CD-NTase-associated protein 14 (Cap14) (16, 18, 33). Applicants expressed the BtCBASS system on the chromosome of Bacillus subtilis PY79 under control of its native promoter (FIG. 2B). Next, this strain and associated negative controls were challenged with phages and plaque formation was measured using a modified double-agar overlay assay. BtCBASS protected B. subtilis by over 104-fold against phages SPP1 and phiB002 as compared to empty vector or BtCBASS expressing a catalytically inactive BtCdnB (BtCBASS^{CD}), but did not protect against phages SPB or phi29 (FIG. 2C-F).

Applicants focused on the interaction with phages SPP1 and phiB002 to further investigate the BtCBASS system and investigated the kinetics of SPP1 restriction by BtCBASS during liquid culture infection. Infection with SPP1 at multiplicities of infection (MOI) of 0.04, or 0.4 did not impact growth of strains expressing BtCBASS (FIG. 2G) and SPP1 was unable to replicate in these conditions as measured by plaque forming units (PFU) (FIG. 2H). In contrast, strains expressing an empty vector or BtCBASS^{CD} decreased in OD₆₀₀ and the PFU of SPP1 increased dramatically indicating successful production of infectious virions (FIG. 2G,H). At an MOI of 4, which ensured that nearly every bacterium was infected, strains expressing BtCBASS decreased in OD₆₀₀ at 60 minutes post infection and did not produce infectious progeny whereas strains harboring an empty vector or BtCBASS^{CD} decreased at 90 minutes post infection and displayed an increase in SPP1 PFUs indicating successful replication (FIG. 2G, H). These results suggested that BtCBASS activates premature cell lysis that prevents completion of the SPP1 replication cycle.

Applicants used fluorescence microscopy to visualize B. subtilis during SPP1 infection at an MOI of 4. At 30 minutes post-infection, bacteria expressing empty vector, BtCBASS, and BtCBASS^{CD} appeared similar. However, at 60 minutes post-infection, bacteria expressing BtCBASS took up propidium iodide and displayed clearly observable signs of membrane disintegration and cellular blebbing as compared to strains expressing empty vector or BtCBASS^{CD} (FIG. 2I-L). By 90 minutes post infection, all strains, including empty vector and BtCBASS^{CD} displayed signs of gross cell lysis (FIG. 2I-J). These data suggest that at 60 minutes BtCBASS initiates a premature cell lysis program that is distinct from the phage-mediated cell lysis that occurs at 90 minutes in susceptible strains under these conditions. These results are consistent with an abortive infection mechanism, in which the antiviral system provides population-level immunity by preventing viral replication in the infected cell through premature cell death or growth arrest (34, 35).

Applicants showed that BtCdnB synthesizes 2′,3′-cGAMP in vitro and Applicants next wanted to confirm that this molecule was also made in vivo, in response to infection. Bacteria expressing empty vector, BtCBASS, and BtCBASS^{CD} were infected with SPP1 or phiB002 at an MOI of 4 and bacteria were harvested and lysed at 50 minutes post infection, prior to BtCBASS-mediated cell lysis. 2′,3′-cGAMP signal increased in bacteria expressing BtCBASS infected with phage and required the catalytic residues of BtCdnB (FIG. 2F). Taken together, these data confirm that in its native biological setting, the BtCBASS system produces 2′,3′-cGAMP in response to phage infection to initiate abortive infection by executing host cell lysis.

### Example 4: Molecular Architecture of Cap14 Bound to 2′,3′-cGAMP

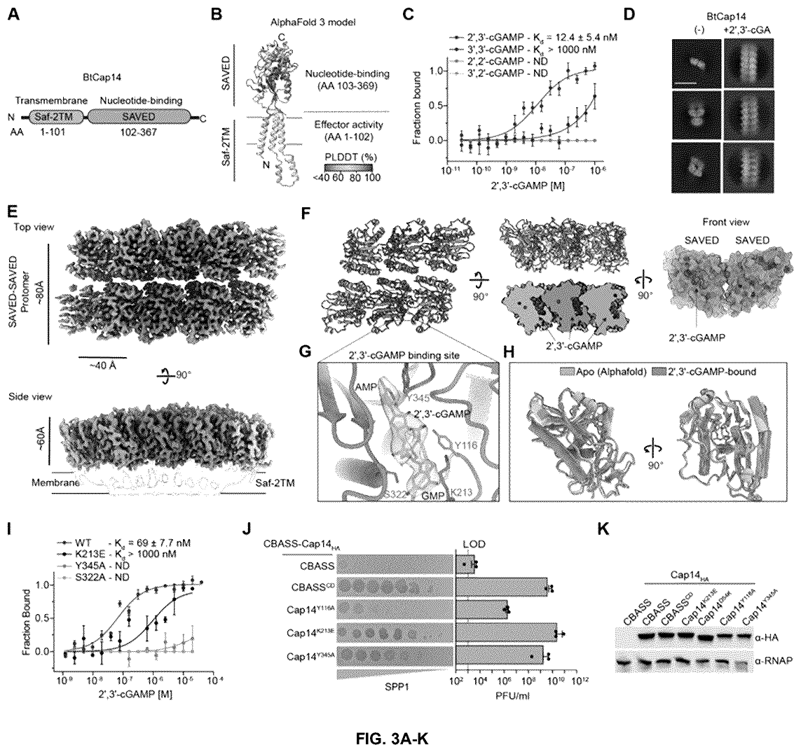

To determine the mechanism for 2′,3′-cGAMP-induced cell death Applicants focused on the Cap14 effector encoded in the BtCBASS operon (BtCap14). BtCap14 encodes an N-terminal SAVED-fused 2™ (Saf-2™) domain that is predicted to have at least 2 transmembrane helices (18). The C-terminus is a globular SMODS-Associated Fused to Various Effector Domains (SAVED) domain. Both predictions were supported by an AlphaFold 3 structure prediction and analysis of the amino acid sequence with TMHMM (FIG. 3A, B, 10A) (18, 36, 37). The Saf-2™ domain does not share homology with any well-characterized proteins, whereas SAVED domains are known to function as cyclic oligonucleotide binding domains in CBASS and Type III CRISPR system effectors where they frequently regulate the activity of a fused enzymatic domain with a defined function (25, 27, 38-40). Saf-2™-SAVED effectors are widespread in bacteria, but the mechanism for Cap14-mediated defense is not known (18).

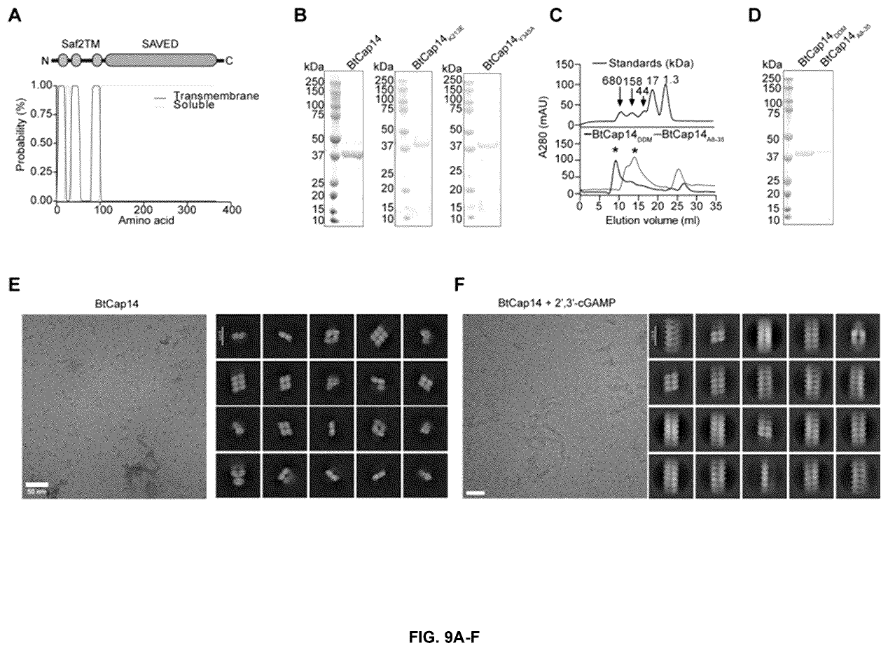

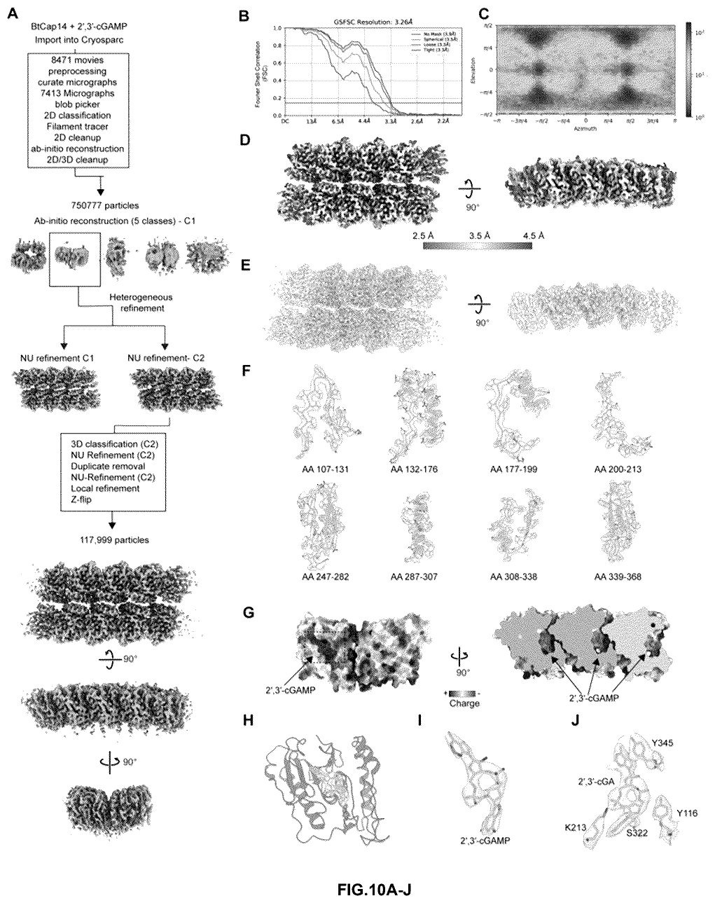

Applicants expressed and purified full-length BtCap14 in detergent micelles and determined that it bound 2′,3′-cGAMP with a K_{d} of 12.4±5.4 nM but did not appreciably bind other cGAMP isoforms (FIG. 3C, 9B). These data confirm that BtCap14 is a direct receptor for 2′,3′-cGAMP. The biochemical function of Saf-2™ domains is not known, thus Applicants opted to visualize BtCap14 using single particle cryo-EM to gain insight into its activation by 2′,3′-cGAMP. BtCap14 was exchanged from detergent into amphipol A8-35 to increase stability, and the elution profile by gel filtration suggested that BtCap14 forms heterogeneous assemblies ranging from monomers (˜42 kDa), dimers, and tetramers (FIGS. 2C-D and FIG. 10). Single particle analysis by Cryo-EM confirmed that in absence of nucleotide, BtCap14 exists in heterogeneous assemblies composed primarily of dimers, with a small fraction of multimer assemblies that stack linearly (FIG. 3D, 9E). When incubated with 2′,3′-cGAMP, BtCap14 formed large filamentous assemblies of parallel-stacked dimers (FIGS. 3D, 9F).

Applicants determined a 3D reconstruction of the BtCap14 SAVED domain multimer bound to 2′,3′-cGAMP that resolved to ˜3.4 Å (FIG. 3E-F, FIG. 10). The BtCap14 multimer consists of two antiparallel monomers forming a dimeric protomer, like what was suggested by 2D classification of the apo state (FIG. 3D, 9). This dimeric protomer further stacks linearly through opposing faces of the SAVED domains above the plane of the membrane where density for the Saf-2™ domain was visible but unable to be resolved to high resolution (FIG. 3E). A single 2′,3′-cGAMP molecule was identified in each individual SAVED domain on opposing faces of the antiparallel dimer at the oligomerization interface between individual protomers (FIG. 3F, G, 10). In the binding pocket, 2′,3′-cGAMP is cradled by aromatic residues Y345 and Y116, which I-stack with the adenosine and guanosine bases (FIG. 3G, 10). The charged residues S322 and K213 each appear to interact with the phosphate backbone from loops on opposing sides, respectively. An overlay of an AlphaFold-3 model of the apo state with our cGAMP-bound structure revealed subtle structural rearrangements between the two states which may represent various folding states of SAVED during ligand-induced multimerization (FIG. 3H).

In support of these findings, mutations K213E, S322A, and Y345A disrupted 2′,3′-cGAMP binding and ablated phage protection by BtCBASS (FIG. 3I, J). Importantly, these mutations did not disrupt protein expression in B. subtilis (FIG. 3K). Collectively, these data indicate that BtCap14 binds 2′,3′-cGAMP which initiates formation of a filamentous assembly that is required for BtCBASS-mediated immunity.

### Example 5: Activated Cap14 Forms Ion Channels in Artificial Membranes

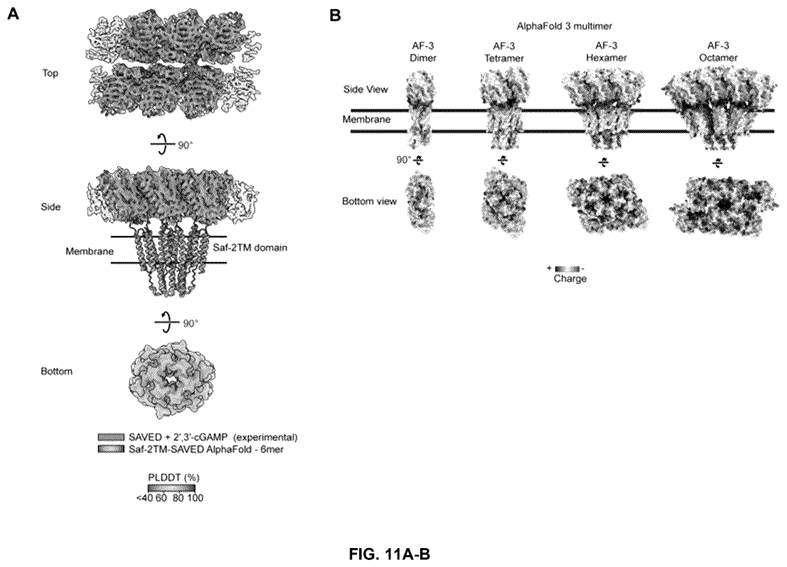

Cyclic oligonucleotide binding to SAVED domains initiates oligomerization, which is required to activate the fused enzymatic effector domains in TIR-SAVED NADases (38), SAVED-CHAT proteases (40), and HNH-SAVED nucleases (27). The Saf-2™ domain does not resemble any domains of known function, but was previously hypothesized to form membrane pores (18). AlphaFold-3 modeling of BtCap14 multimers support this hypothesis and reveal a putative transmembrane pore formed by the Saf-2™ domain that appeared to open in larger assemblies (FIG. 11A, B).

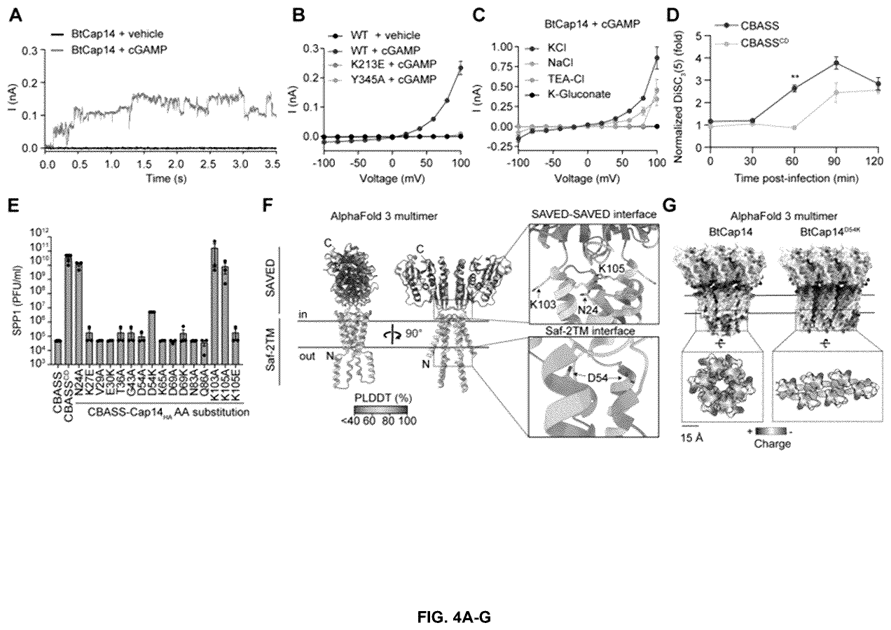

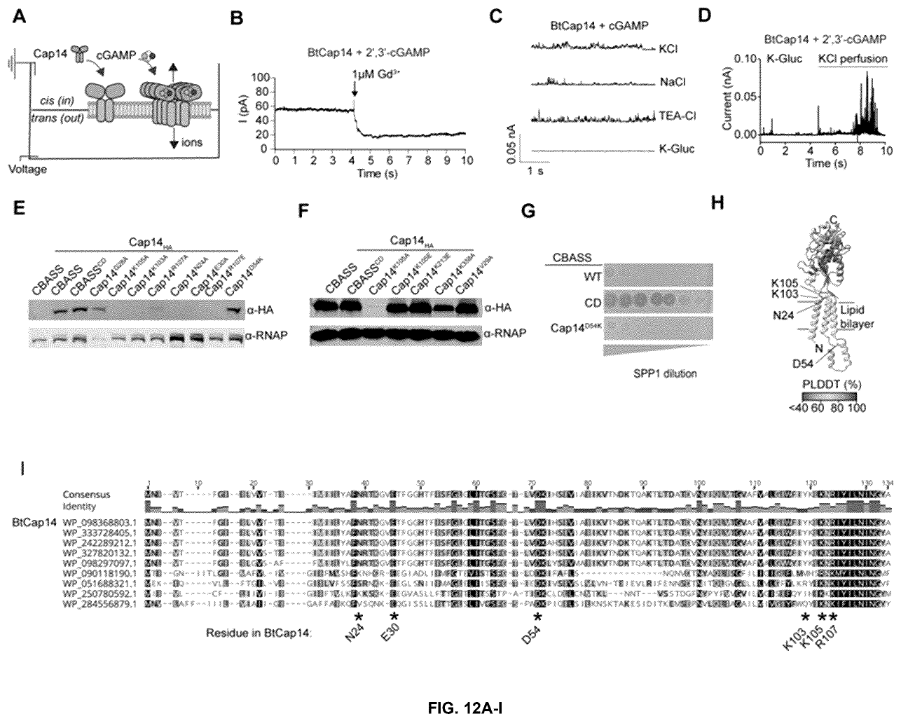

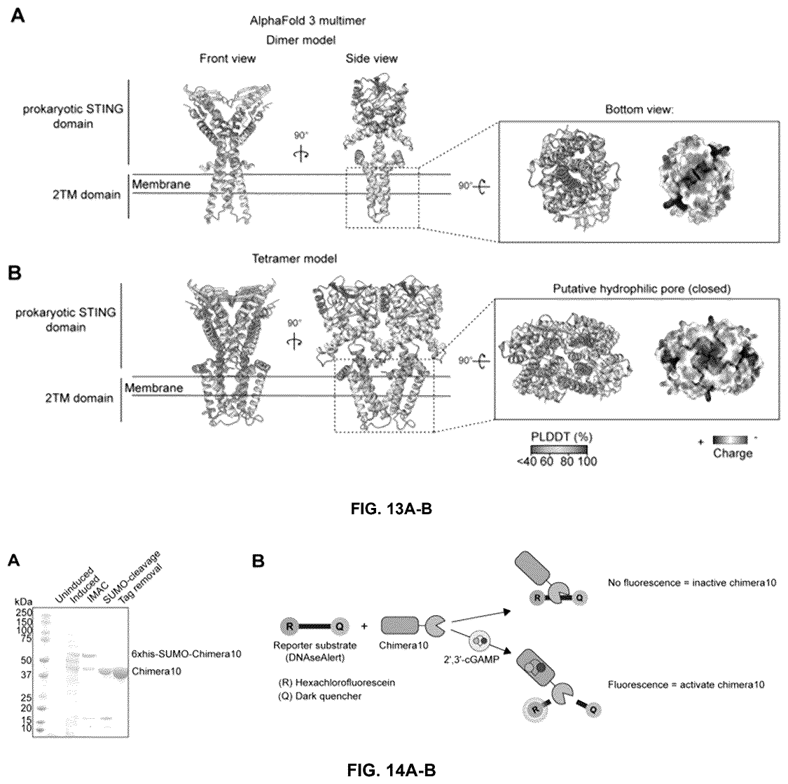

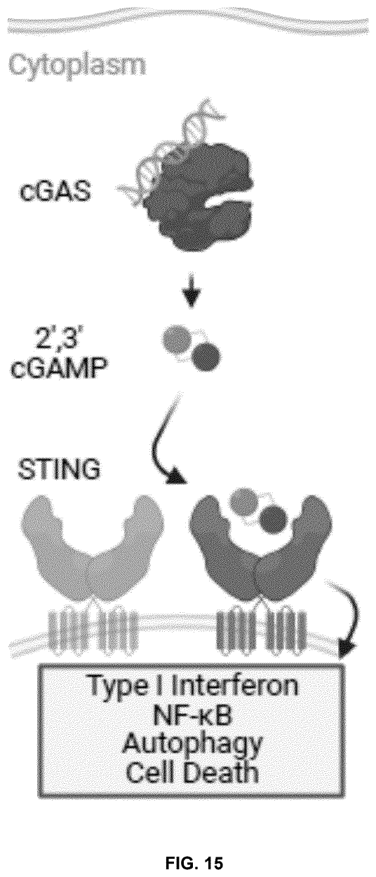

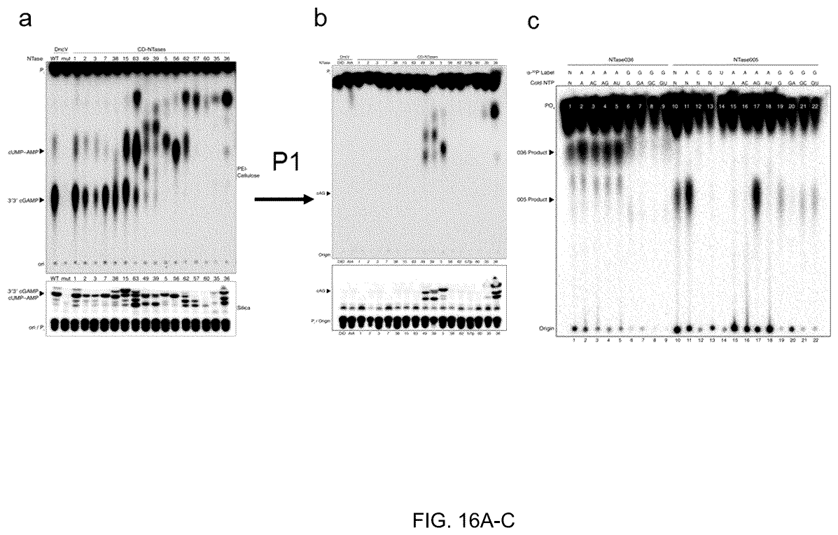

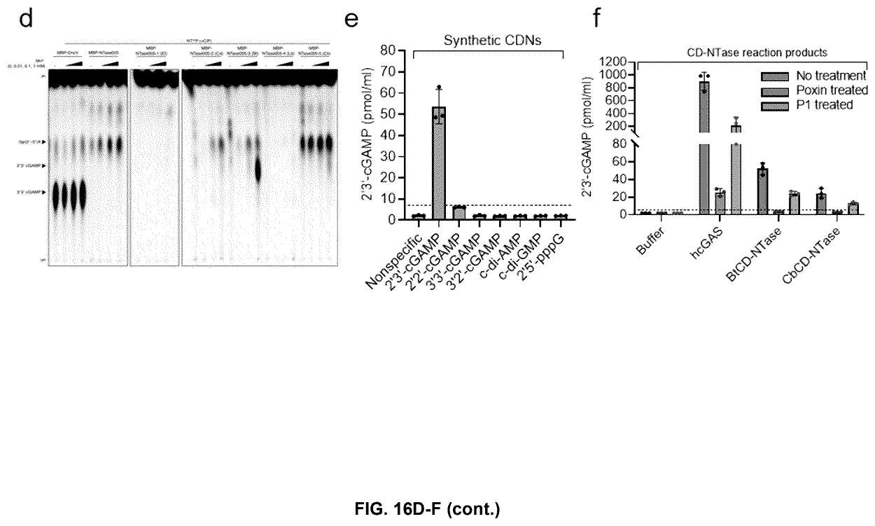

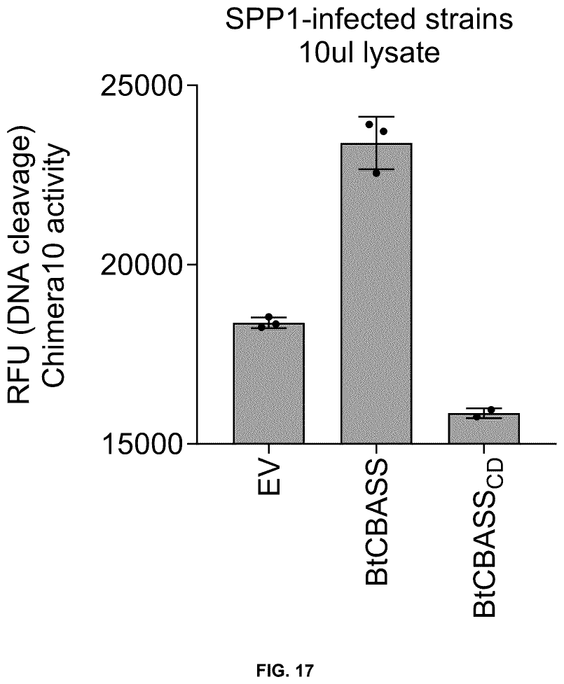

To test whether BtCap14 possesses channel activity, Applicants reconstituted recombinant BtCap14 into artificial lipid bilayers and performed voltage-clamp electrophysiology in symmetric salt solutions of varying composition (+1, 42). In this assay, the voltage (V) generates a driving force across the membrane, and channel-formation is observed via changes in the current (I) due to ion flux (FIG. 12A). Reconstitution of BtCap14 alone did not result in any observable channel activity or membrane disruption, but upon addition of 2′,3′-cGAMP to the cis chamber, Applicants observed stepwise increases in the current trace, the hallmark of channel-forming proteins (FIG. 4A) (+1). Open BtCap14 channels were sensitive to gadolinium chloride (Gd³), an inhibitor of ion channels, as evidenced by the rapid decline in current upon addition (FIG. 12B). The current/voltage (I/V) relationship of BtCap14 when KCl solution was in both the cis and trans chambers confirmed that the channel activity is strictly dependent on 2′,3′-cGAMP binding. Further, BtCap14 variants disrupted for 2′,3′-cGAMP binding did not display channel activity with or without nucleotide (FIG. 4B, 12C).

The current-voltage relationship for BtCap14 shows that channel activity only appears at positive voltages (FIG. 4B). This suggested that BtCap14 displays ion selectivity and/or directionality of transit (42). To test this, Applicants substituted the recording solution in both cis and trans sides with cations and anions of varying sizes and measured BtCap14 activity with cGAMP. The current/voltage profiles and single channel activity were largely unaffected when KCl was replaced with NaCl or tetraethylammonium chloride (TEA-Cl) (FIG. 4C, 12C)). The ionic radius of TEA (˜3.85 Å) is a much larger than K⁺ (˜1.3 Å) or Na⁺ (˜1 Å) and is often impermeable to known cation channels (43). In contrast, when KCl was substituted with potassium gluconate (K-Gluc) Applicants observed a complete lack of channel activity (FIG. 4C, 12C), which could be rescued by buffer exchange of KCl into the cis chamber (FIG. 12D). BtCap14 likely initially inserts into the lipid bilayer in multiple orientations during these assays, however, 2′,3′-cGAMP was only added to the cis chamber and does not cross membranes due to its polar nature. Therefore, only BtCap14 with SAVED domains oriented on the cis side of the bilayer can be activated. Thus, the cis chamber represents the bacterial cytosol, where the SAVED domain would be accessible to 2′,3′-cGAMP produced by CdnB during infection. Our data show ligand-gated, Cl-transport from cis to trans in artificial bilayers, suggesting that BtCap14 channels result in Cl-efflux from bacterial cytosol to the extracellular space during phage infection to execute cell lysis.

BtCap14-mediated Cl-efflux is predicted to depolarize the bacterial membrane. To test this, Applicants measured changes in membrane potential of B. subtilis during SPP1 infection using 3,3′-Dipropylthiadicarbocyanine Iodide (DiSC₃ (5)). DiSC₃ (5) integrates into hyperpolarized membranes, where its fluorescence is quenched. When membranes are depolarized, DiSC₃ (5) is released from the membranes and into the medium resulting in a measurable fluorescence increase. SPP1-infected strains containing BtCBASS increased in signal at 60 minutes post-infection whereas strains expressing BtCBASS^{CD} had no change (FIG. 4D). This is consistent with the onset of ion flux and cell lysis that Applicants observed by OD₆₀₀ and fluorescence microscopy (FIG. 2). By 90 minutes post-infection both strains displayed high DiSC₃ (5) release, which was expected as BtCBASS^{CD} strains undergo phage-mediated lysis.

Applicants attempted to identify amino acid residues in the Saf-2™ domain required for channel activity, but surprisingly most variants selected had no effect on Cap14-mediated protection against SPP1 (FIG. 4E). Select variants within the predicted cytosolic loop between TM1 and TM2 (N24A), and within the predicted linker region between Saf-2™ and the SAVED domain (K103 and K105) resulted in disrupted protein expression (FIG. 12E-F). This was supported by structure-guided AlphaFold-3 modeling of the BtCap14 dimer which predicted these residues to be at interfaces within the core SAVED protomer, highlighting the importance of these linker regions in Cap14 stability (FIG. 4F-G). A single variant: D54K resulted in an intermediate phage protection phenotype against phage SPP1 without disrupting protein expression (FIG. 4E, 12E-G). D54 is located on a predicted extracellular loop (FIG. 4F, G, 12H-I). AlphaFold-3 multimer modeling of the Cap14^{D54K} multimer revealed a constricted assembly in the putative pore (FIG. 4H), thus, Applicants predict that the extracellular loop and D54 are required for full assembly of the putative Saf-2™ channel in the membrane.

### Example 6: STING-Like Effectors Initiate Bacterial Cell Lysis

Our findings that Cap14 is a 2′,3′-cGAMP-gated ion channel motivated us to investigate ion channel properties for other transmembrane proteins involved in antiviral signaling. Applicants focused on STING because recently mammalian STING, which is a transmembrane protein resident in the endoplasmic reticulum, was shown to be a proton channel (+4, 45). Further, metazoan STING forms 2′,3′-cGAMP-induced filaments of stacked dimers (46-49), similar what Applicants observed for BtCap14. Bacteria encode proteins homologous to metazoan STING within CBASS systems (15, 17, 50). The STING-like domain is found fused to Toll/Interleukin-Like Receptor (TIR) domains or to 2™ transmembrane domains. Bacterial TIR-STING effectors bind nucleotide in the STING-like domain, which activates the TIR domain to hydrolyze NAD (17, 51). Bacterial 2™-STING (Cap13) also bound nucleotide, but the effector function is unknown (17). Structure modeling confirmed that Cap13 displayed channel-like architecture with amphipathic arrangements of the 2™ domain and a putative charged central pore (FIG. 13). These structural and functional parallels between Cap14, Cap13, and STING led us to hypothesize that Cap13 also mediates cell lysis to restrict phage.

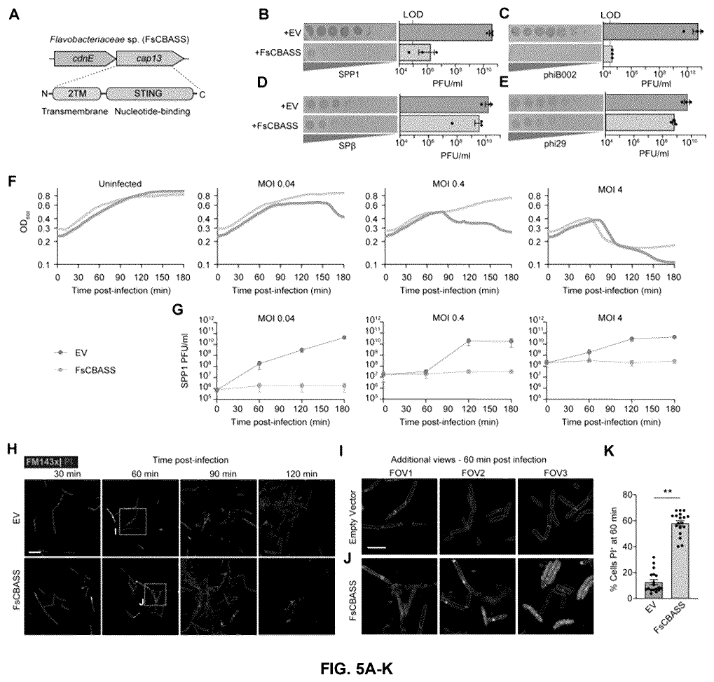

Cap13 is encoded in a type I CBASS system from Flavobacterium (FsCBASS), a two gene operon with CdnE, a CD-NTase that synthesizes cyclic di-guanosine monophosphate (17). Applicants expressed FsCBASS in B. subtilis and found that this operon under its native promoter conferred robust protection against phages SPP1 and phiB002 (FIG. 5A-E). In liquid infection assays, FsCBASS appeared similar to BtCBASS and provided robust defense against phage SPP1 by initiating abortive infection (FIG. 5F-G). At an MOI of 4, FsCBASS displayed a rapid drop in OD₆₀₀ at 60 minutes post infection as compared to an empty vector, and did not produce infectious SPP1 progeny (FIG. 5F-G). Finally, visualization of cell during infection revealed that FsCBASS activation results in cell lysis coupled with significantly increased propidium iodide uptake at 60 minutes post infection with SPP1 compared to the empty vector (FIG. 5H-K). Collectively these data reveal that the programmed cell lysis phenotype extends beyond Cap14 and is also a feature of Cap13 STING-like effectors.

### Example 7: Generation of a 2′,3′-cGAMP Biosensor

An objective of our Application was to identify 2′,3′-cGAMP signaling in bacteria in order to repurpose their components as biological tools. A challenge in the Application of 2′,3′-cGAMP and other cyclic oligonucleotides is the accurate, cost-effective, and rapid quantification of these molecules, which often require laborious methods (52). Applicants reasoned that the 2′,3′-cGAMP-binding SAVED domains Applicants identified might be suitable for use as a biosensor to measure 2′,3′-cGAMP in complex mixtures. However, this domain first needs to be coupled to a readily assayable “output” protein domain.

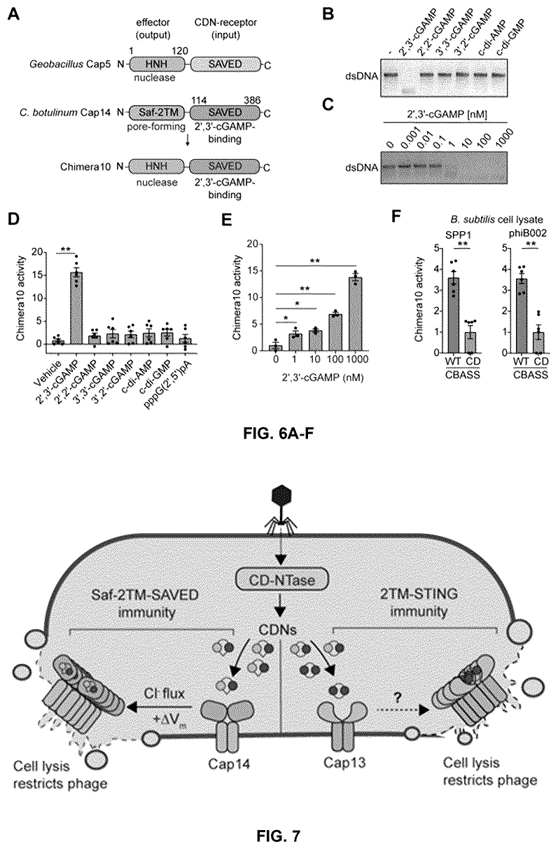

The C-terminal SAVED domain of Cap14 adopts a similar structure to other SAVED domains, which are encoded as a fusions with a wide range of N-terminal enzymatic effector domains (18). Intriguingly, all reported SAVED domain structures, including our own, form nucleotide-activated filaments (27, 38, 40). Therefore, Applicants reasoned that the SAVED domain is modular and could be swapped between CBASS effector proteins to engineer different nucleotide specificity to different enzymatic outputs. Applicants tested this hypothesis by constructing a series of SAVED domain fusions to HNH nucleases, which were selected because nuclease activity is easily analyzed using DNA substrates and reporters. Most of these fusions displayed poor expression or solubility, however, Applicants arrived on a functional chimera between the SAVED domain of CbCap14 and the HNH domain of Parageobacillus thermoglucosidasius Cap5 (GbCap5) called Chimera10 (FIG. 6A, 14A). Chimera10 did not degrade a linear DNA substrate in the absence of nucleotide, however, when incubated with 2′,3′-cGAMP Applicants observed DNA degradation (FIG. 6B). Importantly, no other cGAMP isomers or common cyclic dinucleotides activated DNA cleavage by Chimera10, even at a high concentration of 1 μM, demonstrating the specificity of Chimera10 (FIG. 6B). Chimera10 displayed observable activity using as little as 1 nM 2′,3′-cGAMP and visualizing DNA by agarose-gel electrophoresis (FIG. 6C).

Next, Applicants adapted Chimera 10 for a 96-well microplate assay using a fluorescent DNA oligonucleotide substrate (DNAseAlert) in which substrate cleavage results in an increased fluorescent signal, thus acting as a reporter for nuclease activity (FIG. 14B). Using this approach, Applicants found that Chimera 10 displayed identical specificity for 2′,3′-cGAMP as observed with our agarose-based assays, was not responsive to other cyclic oligonucleotides at 1 μM, and could detect as low as 1 nM of synthetic 2′,3′-cGAMP (FIG. 6D-E). Finally, Applicants tested whether Chimera 10 could be applied to detect 2′,3′-cGAMP in a biological sample. Applicants used cellular lysates of B. subtilis strains expressing BtBCASS and BtCBASS^{CD} infected with SPP1 or phiB002 prepared identically to those used in our earlier ELISA-based measurements. Chimera10 displayed a significant fold-change increase in fluorescence in response to BtCBASS cell lysates compared to the cGAMP-null control (BtCBASS^{CD}) corresponding to ˜1-10 nM of 2′,3′-cGAMP (FIG. 6F), comparable to our measurements by ELISA (FIG. 2I). These data demonstrate proof-of-principle for the use of Chimera10 as a 2′,3′-cGAMP biosensor.

### Example 8: Materials and Methods

### Cloning and Plasmid Construction

All DNA inserts encoding for the indicated proteins or operons were amplified using Q5 polymerase, separated by agarose gel electrophoresis, visualized by SYBR-SAFE, and purified by gel extraction. pLOCO2 vectors (71) constructed for Bacillus subtilis expression used inserts containing SbfI/NotI cut sites, pET vectors for 6×his-SUMO fusion proteins were cloned with BamHI/NotI sites, pET vectors for C-terminal-6×-his tag proteins were cloned with NdeI/BamHI sites (20). Plasmids were assembled via NEB HiFi Assembly using the manufacturers protocol for 1 hour at 50° C. The entire reaction was transformed into chemically competent E. coli OmniPir using heat shock, outgrown in SOC media for 1 hour at 37° C. at 200 RPM, followed by selection on Lysogeny broth (LB) agar supplemented with carbenicillin at 100 μg/ml (Carb100)+1% glucose via incubation overnight at 37° C. Clones were grown in LB+1% glucose+Carb100 100 μg/ml overnight and prepared as freezer stocks with 15% glycerol and stored at −80° C. Clones were validated using colony PCR, plasmids were isolated using the Qiagen Miniprep kit and further validated using Sanger Sequencing (Quintara Biosciences or Genewiz/Azenta) or whole-plasmid sequencing (Plasmidsaurus). Plasmid map construction, primer design, and sequencing analysis was performed using the Geneious Prime software suite.

### Thin Layer Chromatography and PI Endonuclease Treatment.

Thin layer chromatography and radioactive CD-NTase enzymatic assays were performed as described previously (20).

### Bioinformatic Identification of CD-NTases and Phylogenetic Tree Construction.

All CD-NTase numbers and clade assignments are based on the original description from Whiteley et al., Nature 2019 (20). The amino acid sequence for CD-NTase005 (DaCdnB-02) was used as a BLAST seed in Geneious Prime to identify homologous sequences. Hits with an E-value of 1-e⁻¹⁰⁰ or lower and a % Pairwise identity over 40% were selected for a total of 417 AA sequences. A representative species of each unique organism was selected for tree construction and display. These results were then used to generate a global alignment in Geneious Prime with no outgroup, using Jukes-Cantor distance model with neighbor-joining. Species deemed relevant to human health, or agriculture were highlighted, and redundant species were removed for brevity.

### Recombinant Protein Expression and Purification.

MBP-CD-NTase constructs used in FIG. 1a, b and FIG. 8a, c, were purified as described previously (20). For purification of MBP-human cGAS; the plasmid encoding MBP-TEV-hcGAS (pAW1385) was transformed into BL21 (DE3) and selected on LB-agar supplemented with chloramphenicol at 20 μg/ml (Cm20)+1% glucose in an incubator overnight at 37° C. A single colony was grown in 50 ml of LB Cm20 at 37° C. until the optical density at 600 nm (OD₆₀₀) reached 0.5, and then 0.2% arabinose was added to induce expression and the culture was moved to 20° C. for 24 hours. Cells were harvested and lysed as described above and were run over 5 ml of Amylose affinity resin (New England Biolabs), washed with 100 ml of 20 mM sodium phosphate pH 7.2, 500 mM NaCl, 1 mM DTT, and eluted in the same buffer+30 mM maltose monohydrate. Samples were concentrated and stored in 50% glycerol in aliquots at −20° C. until needed.

CD-NTase005 and its homologues were expressed as 6×-his-SUMO fusion proteins in E. coli BL21 Rosetta cells. Plasmids were transformed using heat shock and plated on LB-agar Carb100/Cm20+1% glucose and grown in an overnight at 37° C. A single colony was then used to inoculate a 50 ml starter culture of LB Carb100/Cm20+1% glucose which was grown overnight at 37° C. The next morning, 10 ml of the overnight culture was used to inoculate 1 L of ZYP5052 Studier's Autoinduction media in 2.5 L Thompson flasks (500 ml/flask). Cultures were grown at 37° C. for 8 hours and then switched to 20° C. for 24 hours. Cells were harvested by centrifugation at 4000×g and resuspended in Buffer 1:50 mM sodium phosphate pH 7.2, 500 mM NaCl, 10 mM imidazole, 1% glycerol, 1 mM DTT+1 mg/ml lysozyme. Cells were sonicated on ice at an amplitude of 70, for 30 seconds on/off for 10 minutes total. Sonicated lysates were centrifuged at 14,000×g for 1 hour at 4° C. to pellet debris, and the supernatant was run over 2 ml of Ni-NTa resin (Thermo Fisher Scientific) equilibrated with Buffer 1. The resin was washed with 100 ml of Buffer 2:20 mM sodium phosphate pH 7.2, 500 mM NaCl, 20 mM imidazole, 1 mM DTT to remove nonspecific components bound to the resin. Proteins were eluted using Buffer 3:20 mM sodium phosphate pH 7.2, 500 mM NaCl, 500 mM imidazole, 1 mM DTT. The 6×his-SUMO tag was cleaved using 6×his-ULP1 (produced in-house) via dialysis overnight at 4° C. against 5 L of Buffer 4:20 mM sodium phosphate pH 7.2, 500 mM NaCl, 1 mM DTT using a 10 kDa MWCO dialysis membrane. The cleaved sample was run over 2 ml Ni-NTA to capture cleaved 6×his-SUMO tag and uncleaved protein, whereas the flowthrough containing the recombinant, tag-free CD-NTase was collected. The purified CD-NTase samples were then concentrated using a 3 kDa ultracentrifugation filter at 4000×g at 4° C. to 1 mg/ml and were stored in 50% glycerol at −20° C. until needed. Protein purity was assessed via SDS-PAGE on a 4-20% gel (Genscript) followed by Colloidal Coomassie staining. 6×his-SUMO-VacV Poxin was expressed in BL21 (DE3), and purified and stored identically as described above, but was left as a fusion protein since this does not affect activity. The same protocol was used for expression, purification, and storage of GbCap5 and Chimera10, with the exception that BL21 (DE3) was used as the expression host. Samples were concentrated to ˜2 mg/ml and buffer exchanged into reaction buffer: 10 mM Tris-HCl pH 7.4, 25 mM KCl, 1 mM DTT using a centrifugal filter with a 3 kDa MWCO (Pall Corporation) and experiments were performed immediately to avoid precipitation of Chimera10, and/or the proteins were stored in 50% glycerol at −20° C. which did not affect downstream activity.

For expression of recombinant BtCap14 and its variants; The pAW1517 plasmid was transformed into E. coli C43 competent cells via heat shock and was plated on LB agar+100 μg/ml carbenicillin with 1% glucose and grown overnight at 37° C. The following day a single colony was used to inoculate 50 ml of LB+1% glucose+100 μg/ml carbenicillin, and the culture was grown overnight for 16 hours at 37° C. at 200 RPM. The 50 ml culture was used to inoculate 4 L of ZYP5052 autoinduction medium+1× Vitamin Mix (Teknova)+100 μg/ml carbenicillin (Note: the ZY base medium was modified to include 20 g tryptone, 10 g yeast extract, 5 g NaCl for 1 L). Cultures were split into 2.5 L Thompson flasks (500 ml/flask) and were grown shaking for 8 hours at 37° C., 200 RPM and then lowered to 20° C. and grown for an additional 24 hours at 200 RPM. Cultures were harvested at 4000×g and resuspended in 40 ml lysis buffer per liter of culture (50 mM sodium phosphate, 500 mM NaCl, 1% glycerol, pH 7.0+1 mM PMSF, 2 μl benzonase, +1 complete EDTA-free protease inhibitor tablet (Thermo Fisher Scientific)+1 mg/ml lysozyme. Cells were sonicated on ice as described previously. The sample was centrifuged at 14,000×g for 2 hours at 4° C. and the supernatant was discarded. The pellet was resuspended in 20 ml of lysis buffer+1% n-Dodecyl-B-D-maltoside (DDM/Gold Biosciences)+20 mM imidazole and incubated for 2 hours at 4° C. on an end-over-end rotor to extract membrane proteins. The sample was then centrifuged at 14,000×g for 1 hour at 4° C. to separate cell debris from extracted membrane proteins. The supernatant was run over 5 ml of cobalt resin (Thermo Fisher Scientific) equilibrated with lysis buffer+0.05% DDM+20 mM imidazole in a gravity flow column. The column was washed with 100 ml of 20 mM sodium phosphate, 500 mM NaCl, pH 7.0+0.05% DDM+20 mM imidazole. The protein was eluted in 20 mM sodium phosphate 500 mM NaCl pH 7.4+500 mM imidazole+0.05% DDM and dialyzed with a 10 kDa MWCO dialysis tubing against 20 mM sodium phosphate 500 mM NaCl pH 7.0 overnight at 4° C. BtCap14 variants were purified in the same manner. For gel filtration the sample was concentrated to 500 μl and run over a Superdex 200 column equilibrated with 20 mM sodium phosphate pH 7.0, 500 mM NaCl+0.05% DDM. Sample quality was assessed by SDS-PAGE followed by Colloidal Coomassie staining or via immunoblotting using SDS-PAGE separation followed by transfer to a PVDF membrane using the Bio-Rad Trans-Blot Turbo transfer system, blocking with Odyssey blocking buffer (Bio-Rad) for 1 hour shaking at room temperature, then probing with α-6×-histidine antibody (Rabbit polyclonal, Thermo Scientific Fisher: PA1-983B) at 1:1000 in TBST overnight at 4° C. shaking, followed by 3× washes for 15 minutes each with TBST, followed by secondary detection with Goat-anti-rabbit-680RD in TBST+0.01% SDS for one hour at room temperature, followed by 3× washes with TBST, and resuspension of the membrane in 1×TBS. The blot was then imaged using an Odyssey Imager. Proteins were stored in 50% glycerol at −20° C. until needed.

CD-NTase Enzymatic Assays and Detection of 2′,3′-cGAMP Via ELISA.

1 μg of recombinant CD-NTases/CdnB enzymes or MBP-hcGAS (activated with 100 ng of dsDNA) were incubated at 37° C. for 18 hours with 250 nM ATP and 250 nM GTP in 10 mM Tris-HCl pH 7.4, 25 mM KCl, 20 mM MgCl₂, 1 mM MnCl₂, 1 mM DTT in 200 μl total volume in in triplicate in a 96 well plate sealed with parafilm. Samples were then split in half and one set was treated with recombinant VacV poxin for 2 hours at 37° C. The samples were then analyzed using the 2′,3′-cGAMP ELISA kit (Arbor Assays) via the manufacturer's instructions. The data was analyzed in GraphPad Prism using 4PLC analysis to interpolate the standard curve and unknowns and cross-validated using the manufacturers template. Synthetic cyclic oligonucleotides were used at 50 nM to determine cross-reactivity and agreed with the manufacturer's observations. All synthetic oligonucleotides were obtained from BioLog/Axxora except for 2′,2′-cGAMP, which was obtained from Invivogen. The limit of detection was defined as the highest signal produced by a panel of synthetic cyclic dinucleotide standards and is indicated on the representative graphs, which in this case was 2′,2′-cGAMP. Our results with synthetic nucleotides are consistent with the manufacturers' observations.

Strain Construction in Bacillus subtilis.

Plasmids containing indicated defense systems were transformed into Bacillus subtilis PY79 (hereafter referred to as Bsu) using an integrative vector (pLOCO2) with homology to the 3′ and 5′ ends of the amyE gene and selected on LB+100 μg/ml Spectinomycin (Spec100) overnight at 37° C. Successful transformants were grown overnight in LB+Spec100 and frozen as 15% glycerol stocks for further use. Genomic DNA was isolated from successful transformants using the Qiagen DNAeasy blood and tissue kit and were confirmed for successful integration using PCR.

### Bacteriophage Isolation and Amplification.

Bacillus subtilis phage SPP1 was a kind gift from Daniel Kearns. SPB and phi29 were obtained from the DSMZ stock collection. Phages were plaque purified on Bsu PY79 in LB infection media and mixed with BsuPY79 and amplified on agar plates. Phage was scraped from the plates using a cell scraper and 100 μl of chloroform was added to lyse remaining bacteria, mixed, and allowed to settle. Phage stocks were stored at 4° C. Isolation of the wild Bacillus subtilis phage phiB002 was performed as follows: soil from outside of the University of Colorado Boulder Jennie Smoly Caruthers Biotechnology Building (CU Boulder) was taken and mixed with LB for one hour and then B. subtilis PY79 at log phase (OD₆₀₀=0.4) was added to the mixture and allowed to grow for 3 hours. The mixture was then filter-sterilized and chloroform-treated, and single plaques were isolated on Bsu PY79. The phage phiB002 was isolated, plaque-purified three times, and then stored as described above. All phage stocks were validated using whole genome sequencing (SeqCoast) and were assembled to the reference genomes in Geneious Prime. Phage phiB002 was identified as being related to the myophage SBSphiJ/Grass.

### Bacteriophage Challenge and Plaque Assays on Solid Media.

Bacillus subtilis PY79 (Bsu) strains were grown overnight at 37° C. in LB supplemented with 100 μg/ml spectinomycin (Spec100). Overnight cultures were diluted 1:1000 in Bsu Infection media (LB Lennox formulation+10 mM MgCl₂, 10 mM CaCl₂), and 0.1 mM MnCl₂) and grown in 1 ml volume in a 12 ml culture tube (to allow sufficient aeration), at 30° C. at 200 RPM until mid-log phase (OD₆₀₀=˜ 0.5). 400 μl of Bsu was mixed with 4.5 ml of 0.35% LB Lennox top agar (containing 10 mM MgCl₂, 10 mM CaCl₂), and 0.1 mM MnCl₂ stored at 55° C. until use), inverted, and overlaid on an LB agar plate and allowed to cool for 30 minutes to make the Bsu-containing top-agar. Serial dilutions of the indicated phages were spot-plated, and plates were grown overnight at 30° C. Plaques were imaged and counted the next morning and quantified as plaque-forming units per milliliter (PFU/ml) and displayed as histograms in Graphpad Prism.

### Phage Growth Curve Experiments in Liquid Culture and Viral Titer Measurements.

Bsu strains were grown overnight at 37° C. in LB+Spec100. Strains were back-diluted 1:100 in 25 ml infection medium (LB+10 mM CaCl₂), 10 mM MgCl₂, 0.1 mM MnCl₂) and grown at 37° C. at 200 RPM until an OD₆₀₀ of 0.5-1 was reached. Strains were then normalized by OD₆₀₀ to 0.2 and distributed into a 96 well plate. Phages were added at the indicated multiplicity of infection (MOI) in identical volumes, and the OD₆₀₀ was measured every two minutes using a Tecan Spark plate reader at 30° C. shaking at 200 rpm. For phage replication measurements, samples were taken at the indicated time-point post infection and treated with 1:10 volumes of chloroform to lyse bacteria, and the resulting sample was serially-diluted and spot plated on Bsu PY79 top agar as described above. An increase in titers over the time course was interpreted as successful viral replication and production of infectious progeny, whereas a flatline was interpreted as abortive infection due to a lack of viral replication.

### BtCap14-cGAMP Binding Assays by Microscale Thermophoresis (MST).

MST was performed on a NanoTemper Monolith (University of Colorado Boulder, Biochemistry Shared Instruments pool). BtCap14_{6×his} and related binding variants were diluted from a stock solution into 10 mM HEPES, 1 M KCl, pH 7.2, +0.5% DDM and was labeled using a His-Tag Labeling Kit RED-tris-NTA 2nd Generation Kit (NanoTemper) via the manufacturers protocol in 1.7 ml low-adhesion tubes. The sample was then spun at 16,000×g after labeling to remove aggregates. The samples were prepared with the required serial dilutions of cyclic dinucleotides in PCR tubes and were immediately transferred to Nanotemper glass capillaries and analyzed using the NanoTemper monolith. Replicates were analyzed using the MO-Control software to obtain fraction bound, and the resulting data was plotted using GraphPad Prism. Replicates are defined as three separate MST runs.

### Domain Analysis of BtCap14.

The BtCap14 amino acid sequence was analyzed using (dtu.biolib.com/DeepTMHMM) server (37) and MemSat Software on the PSIPRED server, (bioinf.cs.ucl.ac.uk/psipred/) (72).

### Planar Lipid Bilayer Experiments.

Voltage clamp electrophysiology measurements were performed on an Orbit Mini (Nanion Technologies) horizontal lipid bilayer system and data was collected using Elements Data Reader 3.0. Briefly, using the manufacturers' recommendations: 100 μM MECA chips (Ionera) were bathed in recording solution (150 μl) at 2 nA gain, 1.25 kHz sampling rate at +10 mV. 1,2-diphytanoyl-sn-glycero-3-phosphocholine (DphpC) from Avanti Polar Lipids was dissolved at 10 mg/ml in n-octane (Sigma, electronics grade) and saved at −20° C. until use. Membranes were painted using a paintbrush and Teflon was used to remove excess lipid until the capacitance was between 15-30 pF (100 μM MECA chip) or 30-60 pF (150 μM MECA chip) as per the manufacturer's recommendation. Membrane integrity was monitored for 5 minutes to assess stability. Approximately 10 ng of BtCap14 was added per aperture and each membrane was monitored for fusion spikes to indicate insertion of proteins with the membrane, the fusion pulse option was used to enhance fusion at +40 mV. 1 μM of 2′,3′-cGAMP was added in proximity to the apertures and mixed carefully using a pipette. The current trace was monitored for openings at which point either single channel data was collected at the indicated voltage or current/voltage relationships were determined. Data was analyzed using Elements Data Analyzer 3.0 and plotted in Graphpad Prism. All experiments were conducted at +40 mV unless otherwise indicated. For current/voltage relationships the voltage was clamped at −100 mV and activity was recorded for 10 seconds at 20 mV increments systematically with a return to 0 mV between steps. For substitution measurements experiments were conducted as described above, but for K-Gluconate 10 mM KCl was included in the solution to account for the use of Ag/AgCl₂ electrodes in the experimental setup. For gadolinium chloride inhibition, 1 μM of gadolinium chloride was added at the end of an experiment in which open channels were present.

Membrane Potential Measurements with Disc 3 (5) in B. subtilis

The indicated bacterial strains were grown and infected with phage in a 96 well plate as described earlier. At the indicated timepoints, Disc3 (5) was added to a final concentration of 5 μM (from a 500 M stock solution dissolved in DMSO) and allowed to equilibrate for 2 minutes. Fluorescence intensity was measured on a Tecan SPARK fluorescence plate reader at 620/670±5 nm Ex/Em.

### Chimera10 Nuclease Assays

Linear DNA products were amplified by PCR to generate template. 1 μg of Chimera10 was incubated with 1 μM of the indicated cyclic dinucleotide with 500 ng template in a final buffer of 10 mM Tris-HCl pH 7.4, 25 mM KCl, 1 mM DTT for 1 hour at 37° C. DNA degradation was visualized on 1% agarose gels stained with SYBR-Safe. Data was representative of n>3 protein preparations and n>3 biological replicates.

### DNAse Alert Assay for Chimera10 Nuclease Activity

DNAse Alert was purchased from IDT. One tube was resuspended in 5 ml of reaction buffer (as described in previous section). In a 96 well plate, samples were prepared in 100 μl buffer+substrate with the indicated cyclic dinucleotide stock (10 μl volume was added total)+3 μg of Chimera10. The reaction was monitored at an excitation/emission of 360/460 nm in a TECAN Spark plate reader at 37° C. Data was normalized as a fold change/vehicle to account for variability between assays. Data was plotted as fold-change over the vehicle (no nucleotide for synthetic standards) or against the cGAMP-null strain in the case of cell lysates to account for background.

### Fluorescence Microscopy.

A spinning disk microscope (Nikon Eclipse Ti/Yokogawa CSU-X1) was used to image the cells. Samples were plated on a 4-chamber slide (Ibidi μ-slide) below a 1.5% agarose pad. At least nine locations were captured for each sample. For each location, three images were captured using the following optical configurations: DAPI (405 nm ex./428-466 nm em.; 200 ms exposure; 10% power), GFP (488 nm ex./500-550 nm em.; 150 ms exposure; 10% power), and TRITC (561 nm ex./575-623 nm em.; 300 ms exposure; 20% power). Note that the exposure time and excitation power was varied from day-to-day to ensure that the signal-to-noise ratio was consistent. Images were analyzed in Fiji to count cells that were positive for propidium iodide. Data was representative of n=3 separate experiments.

### Cryo-EM Sample Preparation

BtCap14_{6×his} in 0.05% DDM was incubated with 25 mg of A8-35 amphipols with Biobeads overnight on an end-over-end rotor. The exchanged sample was separated on a Superdex 200 column and the peak fraction between ˜44 and ˜158 kDa was pooled, concentrated to 1 mg/ml and vitrified on glow-discharged Quantifoil copper 400 mesh R1.2/1.3 using an FEI Vitrobot Mark IV at 100% humidity, 5° C., 8 seconds blotting time, blotting force 1.

### CryoEM Data Collection and Processing

BtCap14+2′,3′-cGAMP datasets were collected on an FEI Titan Krios G3 (CU BioKEM Cryo-electron microscopy facility) using the EPU software (Thermo Fisher Scientific) in EER format at 0.97 Å/pix, 50 E/Å² total dose, 7.85 s exposure, frames: 270 with defocus values ranging from −0.3 to −2.5 μM. All processing was performed in CryoSparc v4.4. For the BtCap14+cGAMP dataset: 8471 micrographs were preprocessed using patch motion correction, patch CTF, and then curated to remove movies with poor ice thickness or CTF fits worse than 7 Å yielding 7413 movies. The blob picker was used for initial particle picking followed by particle extraction with a box size of 540, iterative 2D classification and template-based particle picking. 2D inputs were then used as templates for the filament tracer tool, followed by 2D classification, cleanup, ab-initio model generation (C1), heterogeneous refinements, non-uniform refinement (C2 symmetry applied) and 3D cleanup (C2), followed by non-uniform refinement and local refinement. For the BtCap14 apo state, 8415 movies were collected, and preprocessed identically to yield 7454 micrographs of suitable quality. The blob picker was used for particle picking followed by 2D classification on particles extracted with a box size of 540. Subsequent attempts at 3D reconstruction were unsuccessful.

### Atomic Model Building

Model Building was performed ab-initio in Phenix to generate an initial template, and then further built using Coot. Ligand coordinates for 2′,3′-cGAMP (1SY) were imported into Phenix to generate restraints using Elbow and were built into the density map followed close inspection of the density map, followed by iterative real-space refinement and manual fixing of outliers in Coot. Atomic model and map visualization was performed in UCSF ChimeraX (73).

### Accession Numbers

The accession numbers for genes: DaCdnB, WP_031517737.1; BtCdnB, EEM25276.1; CbCdnB, WP_053342861.1; BtCap14, WP_098368803.1; CbCap14, WP_053342862.1; GbCap5; WP_013400843.1, all of which are incorporated herein by reference.

### REFERENCES

1. K. P. Hopfner, V. Hornung, Molecular mechanisms and cellular functions of cGAS-STING signaling. Nat Rev Mol Cell Biol 21, 501-521 (2020).
2. H. Ishikawa, G. N. Barber, STING is an endoplasmic reticulum adaptor that facilitates innate immune signalling. Nature 455, 674-678 (2008).
3. X. Gui, H. Yang, T. Li, X. Tan, P. Shi, M. Li, F. Du, Z. J. Chen, Autophagy induction via STING trafficking is a primordial function of the cGAS pathway. Nature 567, 262-266 (2019).
4. A. Decout, J. D. Katz, S. Venkatraman, A. Ablasser, The cGAS-STING pathway as a therapeutic target in inflammatory diseases. Nat Rev Immunol 21, 548-569 (2021).
5. J. H. Maltback, S. Cambier, J. M. Snyder, D. B. Stetson, ABCC1 transporter exports the immunostimulatory cyclic dinucleotide cGAMP. Immunity 55, 1799-1812.e4 (2022).
6. L. J. Lahey, R. E. Mardjuki, X. Wen, G. T. Hess, C. Ritchie, J. A. Carozza, V. Bohnert, M. Maduke, M. C. Bassik, L. Li, LRRC8A: C/E Heteromeric Channels Are Ubiquitous Transporters of cGAMP. Molecular Cell 80, 578-591.e5 (2020).
7. J. A. Carozza, V. Bohnert, K. C. Nguyen, G. Skariah, K. E. Shaw, J. A. Brown, M. Rafat, R. von Eyben, E. E. Graves, J. S. Glenn, M. Smith, L. Li, Extracellular cGAMP is a cancer-cell-produced immunotransmitter involved in radiation-induced anticancer immunity. Nat Cancer 1, 184-196 (2020).
8. C. Ritchie, A. F. Cordova, G. T. Hess, M. C. Bassik, L. Li, SLC19A1 Is an Importer of the Immunotransmitter cGAMP. Molecular Cell 75, 372-381.e5 (2019).
9. A. F. Cordova, C. Ritchie, V. Bohnert, L. Li, Human SLC46A2 Is the Dominant cGAMP Importer in Extracellular cGAMP-Sensing Macrophages and Monocytes. ACS Cent. Sci. 7, 1073-1088 (2021).
10. C. Zhou, X. Chen, R. Planells-Cases, J. Chu, L. Wang, L. Cao, Z. Li, K. I. López-Cayuqueo, Y. Xie, S. Ye, X. Wang, F. Ullrich, S. Ma, Y. Fang, X. Zhang, Z. Qian, X. Liang, S. Q. Cai, Z. Jiang, D. Zhou, Q. Leng, T. S. Xiao, K. Lan, J. Yang, H. Li, C. Peng, Z. Qiu, T. J. Jentsch, H. Xiao, Transfer of cGAMP into Bystander Cells via LRRC8 Volume-Regulated Anion Channels Augments STING-Mediated Interferon Responses and Anti-viral Immunity. Immunity 52, 767-781.e6 (2020).
11. A. Bridgeman, J. Maelfait, T. Davenne, T. Partridge, Y. Peng, A. Mayer, T. Dong, V. Kaever, P. Borrow, J. Rehwinkel, Viruses transfer the antiviral second messenger cGAMP between cells. Science 349, 1228-1232 (2015).
12. D. L. Burdette, K. M. Monroe, K. Sotelo-Troha, J. S. Iwig, B. Eckert, M. Hyodo, Y. Hayakawa, R. E. Vance, STING is a direct innate immune sensor of cyclic di-GMP. Nature 478, 515-518 (2011).
13. Q. Yin, Y. Tian, V. Kabaleeswaran, X. Jiang, D. Tu, M. J. Eck, Z. J. Chen, H. Wu, Cyclic di-GMP Sensing via the Innate Immune Signaling Protein STING. Molecular Cell 46, 735-745 (2012).
14. J. J. Woodward, A. T. Iavarone, D. A. Portnoy, c-di-AMP Secreted by Intracellular Listeria monocytogenes Activates a Host Type I Interferon Response. Science 328, 1703-1705 (2010).
15. D. Cohen, S. Melamed, A. Millman, G. Shulman, Y. Oppenheimer-Shaanan, A. Kacen, S. Doron, G. Amitai, R. Sorek, Cyclic GMP-AMP signalling protects bacteria against viral infection. Nature 574, 691-695 (2019).
16. A. Millman, S. Melamed, G. Amitai, R. Sorek, Diversity and classification of cyclic-oligonucleotide-based anti-phage signalling systems. Nat Microbiol 5, 1608-1615 (2020).
17. B. R. Morehouse, A. A. Govande, A. Millman, A. F. A. Keszei, B. Lowey, G. Ofir, S. Shao, R. Sorek, P. J. Kranzusch, STING cyclic dinucleotide sensing originated in bacteria. Nature 586, 429-433 (2020).
18. A. M. Burroughs, D. Zhang, D. E. Schäffer, L. M. Iyer, L. Aravind, Comparative genomic analyses reveal a vast, novel network of nucleotide-centric systems in biological conflicts, immunity and signaling. Nucleic Acids Res 43, 10633-10654 (2015).
19. Q. Ye, R. K. Lau, I. T. Mathews, E. A. Birkholz, J. D. Watrous, C. S. Azimi, J. Pogliano, M. Jain, K. D. Corbett, HORMA Domain Proteins and a Trip13-like ATPase Regulate Bacterial cGAS-like Enzymes to Mediate Bacteriophage Immunity. Molecular Cell 77, 709-722.e7 (2020).
20. A. T. Whiteley, J. B. Eaglesham, C. C. de Oliveira Mann, B. R. Morehouse, B. Lowey, E. A. Nieminen, O. Danilchanka, D. S. King, A. S. Y. Lee, J. J. Mekalanos, P. J. Kranzusch, Bacterial cGAS-like enzymes synthesize diverse nucleotide signals. Nature 567, 194-199 (2019).
21. B. W. Davies, R. W. Bogard, T. S. Young, J. J. Mekalanos, Coordinated Regulation of Accessory Genetic Elements Produces Cyclic Di-Nucleotides for V. cholerae Virulence. Cell 149, 358-370 (2012).
22. B. Duncan-Lowey, P. J. Kranzusch, CBASS phage defense and evolution of antiviral nucleotide signaling. Current Opinion in Immunology 74, 156-163 (2022).
23. K. M. Slavik, P. J. Kranzusch, CBASS to cGAS-STING: The Origins and Mechanisms of Nucleotide Second Messenger Immune Signaling. Annu. Rev. Virol. 10, annurev-virology-111821-115636 (2023).
24. G. B. Severin, M. S. Ramliden, K. C. Ford, A. J. Van Alst, R. Sanath-Kumar, K. A. Decker, B. Y. Hsuch, G. Chen, S. H. Yoon, L. M. Demey, B. J. O'Hara, C. R. Rhoades, V. J. DiRita, W. L. Ng. C. M. Waters, Activation of a Vibrio cholerae CBASS anti-phage system by quorum sensing and folate depletion. mBio 14, e00875-23 (2023).
25. S. Fatma, A. Chakravarti, X. Zeng, R. H. Huang, Molecular mechanisms of the CdnG-Cap5 antiphage defense system employing 3′,2′-cGAMP as the second messenger. Nat Commun 12, 6381 (2021).
26. A. A. Govande, B. Duncan-Lowey, J. B. Eaglesham, A. T. Whiteley, P. J. Kranzusch, Molecular basis of CD-NTase nucleotide selection in CBASS anti-phage defense. Cell Reports 35 (2021).
27. B. Lowey, A. T. Whiteley, A. F. A. Keszei, B. R. Morehouse, I. T. Mathews, S. P. Antine, V. J. Cabrera, D. Kashin, P. Niemann, M. Jain, F. Schwede, J. J. Mekalanos, S. Shao, A. S. Y. Lee, P. J. Kranzusch, CBASS Immunity Uses CARF-Related Effectors to Sense 3′-5′- and 2′-5′-Linked Cyclic Oligonucleotide Signals and Protect Bacteria from Phage Infection. Cell 182, 38-49.e17 (2020).
28. P. Gao, M. Ascano, Y. Wu, W. Barchet, B. L. Gaffney, T. Zillinger, A. A. Serganov, Y. Liu, R. A. Jones, G. Hartmann, T. Tuschl, D. J. Patel, Cyclic [G (2′,5′) pA (3′,5′) p] Is the Metazoan Second Messenger Produced by DNA-Activated Cyclic GMP-AMP Synthase. Cell 153, 1094-1107 (2013).
29. E. Balint, I. Unk, For the Better or for the Worse? The Effect of Manganese on the Activity of Eukaryotic DNA Polymerases. International Journal of Molecular Sciences 25, 363 (2024).
30. Z. Zhao, Z. Ma. B. Wang, Y. Guan, X. D. Su, Z. Jiang, Mn2+ Directly Activates cGAS and Structural Analysis Suggests Mn2+ Induces a Noncanonical Catalytic Synthesis of 2′3′-cGAMP. Cell Reports 32, 108053 (2020).
31. J. B. Eaglesham, Y. Pan, T. S. Kupper, P. J. Kranzusch, Viral and metazoan poxins are cGAMP-specific nucleases that restrict cGAS-STING signalling. Nature 566, 259-263 (2019).
32. J. B. Eaglesham, K. L. McCarty, P. J. Kranzusch, Structures of diverse poxin cGAMP nucleases reveal a widespread role for cGAS-STING evasion in host-pathogen conflict. eLife 9, e59753 (2020).
33. B. Duncan-Lowey, N. K. McNamara-Bordewick, N. Tal, R. Sorek, P. J. Kranzusch, Effector-mediated membrane disruption controls cell death in CBASS antiphage defense. Molecular Cell 81, 5039-5051.e5 (2021).
34. A. Lopatina, N. Tal, R. Sorek, Abortive Infection: Bacterial Suicide as an Antiviral Immune Strategy. Annu. Rev. Virol. 7, 371-384 (2020).
35. N. Aframian, A. Eldar, Abortive infection antiphage defense systems: separating mechanism and phenotype. Trends in Microbiology, doi: 10.1016/j.tim.2023.05.002 (2023).
36. J. Abramson, J. Adler, J. Dunger, R. Evans, T. Green, A. Pritzel, O. Ronneberger, L. Willmore, A. J. Ballard, J. Bambrick, S. W. Bodenstein, D. A. Evans, C. C. Hung. M. O'Neill, D. Reiman, K. Tunyasuvunakool, Z. Wu, A. Žemgulytė, E. Arvaniti, C. Beattie, O. Bertolli, A. Bridgland, A. Cherepanov, M. Congreve, A. I. Cowen-Rivers, A. Cowie, M. Figurnov, F. B. Fuchs, H. Gladman, R. Jain, Y. A. Khan, C. M. R. Low, K. Perlin, A. Potapenko, P. Savy, S. Singh, A. Stecula, A. Thillaisundaram, C. Tong, S. Yakneen, E. D. Zhong, M. Zielinski, A. Žídek, V. Bapst, P. Kohli, M. Jaderberg, D. Hassabis, J. M. Jumper, Accurate structure prediction of biomolecular interactions with AlphaFold 3. Nature 630, 493-500 (2024).
37. J. Hallgren, K. D. Tsirigos, M. D. Pedersen, J. J. A. Armenteros, P. Marcatili, H. Nielsen, A. Krogh, O. Winther, DeepTMHMM predicts alpha and beta transmembrane proteins using deep neural networks. bioRxiv [Preprint] (2022). doi.org/10.1101/2022.04.08.487609.
38. G. Hogrel, A. Guild, S. Graham, H. Rickman, S. Grüschow, Q. Bertrand, L. Spagnolo, M. F. White, Cyclic nucleotide-induced helical structure activates a TIR immune effector. Nature 608, 808-812 (2022).
39. O. Rechkoblit, D. Sciaky, D. F. Kreitler, A. Buku, J. Kottur, A. K. Aggarwal, Activation of CBASS Cap5 endonuclease immune effector by cyclic nucleotides. Nat Struct Mol Biol 31, 767-776 (2024).
40. J. A. Steens, J. P. K. Bravo, C. R. P. Salazar, C. Yildiz, A. M. Amieiro, S. Kostlbacher, S. H. P. Prinsen, A. S. Andres, C. Patinios, A. Bardis, A. Barendregt, R. A. Scheltema, T. J. G. Ettema, J. van der Oost, D. W. Taylor, R. H. J. Staals, Type III-B CRISPR-Cas cascade of proteolytic cleavages. Science 383, 512-519 (2024).
41. E. Zaitseva, A. Obergrussberger, C. Weichbrodt, M. Boukhet, F. Bernhard, C. Hein, G. Baaken, N. Fertig, J. C. Behrends, “Electrophysiology on Channel-Forming Proteins in Artificial Lipid Bilayers: Next-Generation Instrumentation for Multiple Recordings in Parallel” in Patch Clamp Electrophysiology: Methods and Protocols, M. Dallas, D. Bell, Eds. (Springer U S, New York, NY, 2021: doi.org/10.1007/978-1-0716-0818-0_4) Methods in Molecular Biology, pp. 67-92.
42. R. B. Stockbridge, M. F. Tsai, “Chapter Eighteen-Lipid Reconstitution and Recording of Recombinant Ion Channels” in Methods in Enzymology, A. K. Shukla, Ed. (Academic Press, 2015; sciencedirect.com/science/article/pii/S0076687914001426) vol. 556 of Membrane Proteins—Production and Functional Characterization, pp. 385-404.
43. G. Bi, M. Su, N. Li, Y. Liang, S. Dang, J. Xu, M. Hu, J. Wang, M. Zou, Y. Deng, Q. Li, S. Huang, J. Li, J. Chai, K. He, Y. Chen, J. M. Zhou, The ZARI resist some is a calcium-permeable channel triggering plant immune signaling. Cell 184, 3528-3541.e12 (2021).
44. B. Liu, R. J. Carlson, I. S. Pires, M. Gentili, E. Feng, Q. Hellier, M. A. Schwartz, P. C. Blainey, D. J. Irvine, N. Hacohen, Human STING is a proton channel. Science 381, 508-514 (2023).
45. J. Xun, Z. Zhang, B. Lv, D. Lu, H. Yang, G. Shang, J. X. Tan, A conserved ion channel function of STING mediates noncanonical autophagy and cell death. EMBO reports 25, 544-569 (2024).
46. G. Shang, C. Zhang, Z. J. Chen, X. Bai, X. Zhang, Cryo-EM structures of STING reveal its mechanism of activation by cyclic GMP-AMP. Nature 567, 389-393 (2019).
47. D. Lu, G. Shang, J. Li, Y. Lu, X. Bai, X. Zhang, Activation of STING by targeting a pocket in the transmembrane domain. Nature 604, 557-562 (2022).
48. J. Li, S. M. Canham, H. Wu, M. Henault, L. Chen, G. Liu, Y. Chen, G. Yu, H. R. Miller, V. Hornak, S. M. Brittain, G. A. Michaud, A. Tutter, W. Broom, M. E. Digan, S. M. McWhirter, K. E. Sivick, H. T. Pham, C. H. Chen, G. S. Tria, J. M. McKenna, M. Schirle, X. Mao, T. B. Nicholson, Y. Wang, J. L. Jenkins, R. K. Jain, J. A. Tallarico, S. J. Patel, L. Zheng, N. T. Ross, C. Y. Cho, X. Zhang, X. C. Bai, Y. Feng, Activation of human STING by a molecular glue-like compound. Nat Chem Biol 20, 365-372 (2024).
49. S. Liu, B. Yang, Y. Hou, K. Cui, X. Yang, X. Li, L. Chen, S. Liu, Z. Zhang, Y. Jia, Y. Xie, Y. Xue, X. Li, B. Yan, C. Wu, W. Deng, J. Qi, D. Lu, G. F. Gao, P. Wang, G. Shang, The mechanism of STING autoinhibition and activation. Molecular Cell 83, 1502-1518.e10 (2023).
50. A. M. Burroughs, L. Aravind, Identification of Uncharacterized Components of Prokaryotic Immune Systems and Their Diverse Eukaryotic Reformulations. J Bacteriol 202, e00365-20/jb/202/24/JB.00365-20.atom (2020).
51. B. R. Morehouse, M. C. J. Yip, A. F. A. Keszei, N. K. McNamara-Bordewick, S. Shao, P. J. Kranzusch, Cryo-EM structure of an active bacterial TIR-STING filament complex. Nature 608, 803-807 (2022).
52. K. M. Garland, T. L. Sheehy, J. T. Wilson, Chemical and Biomolecular Strategies for STING Pathway Activation in Cancer Immunotherapy. Chem. Rev. 122, 5977-6039 (2022).
53. S. F. Erttmann, P. Swacha, K. M. Aung, B. Brindefalk, H. Jiang, A. Hartlova, B. E. Uhlin, S. N. Wai, N. O. Gekara, The gut microbiota prime systemic antiviral immunity via the cGAS-STING-IFN-I axis. Immunity 55, 847-861.e10 (2022).
54. Y. Choi, J. N. Lichterman, L. A. Coughlin, N. Poulides, W. Li, P. Del Valle, S. N. Palmer, S. Gan, J. Kim, X. Zhan, Y. Gao, B. M. Evers, L. V. Hooper, C. Pasare, A. Y. Koh, Immune checkpoint blockade induces gut microbiota translocation that augments extraintestinal antitumor immunity. Science Immunology 8, eabo2003 (2023).
55. E. N. Baruch, I. Youngster, G. Ben-Betzalel, R. Ortenberg, A. Lahat, L. Katz, K. Adler, D. Dick-Necula, S. Raskin, N. Bloch, D. Rotin, L. Anafi, C. Avivi, J. Melnichenko, Y. Steinberg-Silman, R. Mamtani, H. Harati, N. Asher, R. Shapira-Frommer, T. Brosh-Nissimov, Y. Eshet, S. Ben-Simon, O. Ziv, M. A. W. Khan, M. Amit, N. J. Ajami, I. Barshack, J. Schachter, J. A. Wargo, O. Koren, G. Markel, B. Boursi, Fecal microbiota transplant promotes response in immunotherapy-refractory melanoma patients. Science 371, 602-609 (2021).
56. L. Zitvogel, Y. Ma, D. Raoult, G. Kroemer, T. F. Gajewski, The microbiome in cancer immunotherapy: Diagnostic tools and therapeutic strategies. Science 359, 1366-1370 (2018).
57. R. D. Luteijn, S. A. Zaver, B. G. Gowen, S. K. Wyman, N. E. Garelis, L. Onia, S. M. McWhirter, G. E. Katibah, J. E. Corn, J. J. Woodward, D. H. Raulet, SLC19A1 transports immunoreactive cyclic dinucleotides. Nature 573, 434-438 (2019).
58. X. Zhang, H. Shi, J. Wu, X. Zhang, L. Sun, C. Chen, Z. J. Chen, Cyclic GMP-AMP Containing Mixed Phosphodiester Linkages Is An Endogenous High-Affinity Ligand for STING. Molecular Cell 51, 226-235 (2013).
59. N. Tal, B. R. Morehouse, A. Millman, A. Stokar-Avihail, C. Avraham, T. Fedorenko, E. Yirmiya, E. Herbst, A. Brandis, T. Mehlman, Y. Oppenheimer-Shaanan, A. F. A. Keszei, S. Shao, G. Amitai, P. J. Kranzusch, R. Sorek, Cyclic CMP and cyclic UMP mediate bacterial immunity against phages. Cell 184, 5728-5739.e16 (2021).
60. L. Payne, S. Jackson, R. Pinilla-Redondo, Supramolecular assemblies in bacterial immunity: an emerging paradigm. Trends in Microbiology 32, 828-831 (2024).
61. C. F. Baca, Y. Yu, J. T. Rostol, P. Majumder, D. J. Patel, L. A. Marraffini, The CRISPR effector Cam1 mediates membrane depolarization for phage defence. Nature 625, 797-804 (2024).
62. A. R. VanderWal, J. U. Park, B. Polevoda, J. K. Nicosia, A. M. Molina Vargas, E. H. Kellogg, M. R. O'Connell, Csx28 is a membrane pore that enhances CRISPR-Cas 13b-dependent antiphage defense. Science 380, 410-415 (2023).
63. J. Ding, K. Wang, W. Liu, Y. She, Q. Sun, J. Shi, H. Sun, D. C. Wang, F. Shao, Pore-forming activity and structural autoinhibition of the gasdermin family. Nature 535, 111-116 (2016).
64. A. G. Johnson, T. Wein, M. L. Mayer, B. Duncan-Lowey, E. Yirmiya, Y. Oppenheimer-Shaanan, G. Amitai, R. Sorek, P. J. Kranzusch, Bacterial gasdermins reveal an ancient mechanism of cell death. Science 375, 221-225 (2022).
65. J. Ruan, S. Xia, X. Liu, J. Lieberman, H. Wu, Cryo-EM structure of the gasdermin A3 membrane pore. Nature 557, 62-67 (2018).
66. M. Degen, J. C. Santos, K. Pluhackova, G. Cebrero, S. Ramos, G. Jankevicius, E. Hartenian, U. Guillerm, S. A. Mari, B. Kohl, D. J. Müller, P. Schanda, T. Maier, C. Perez, C. Sieben, P. Broz, S. Hiller, Structural basis of NINJ1-mediated plasma membrane rupture in cell death. Nature 618, 1065-1071 (2023).
67. N. Kayagaki, O. S. Kornfeld, B. L. Lee, I. B. Stowe, K. O'Rourke, Q. Li, W. Sandoval, D. Yan, J. Kang, M. Xu, J. Zhang, W. P. Lee, B. S. Mckenzie, G. Ulas, J. Payandeh, M. Roose-Girma, Z. Modrusan, R. Reja, M. Sagolla, J. D. Webster, V. Cho, T. D. Andrews, L. X. Morris, L. A. Miosge, C. C. Goodnow, E. M. Bertram, V. M. Dixit, NINJ1 mediates plasma membrane rupture during lytic cell death. Nature 591, 131-136 (2021).
68. A. J. Pollock, S. A. Zaver, J. J. Woodward, A STING-based biosensor affords broad cyclic dinucleotide detection within single living eukaryotic cells. Nat Commun 11, 3533 (2020).
69. D. Bose, Y. Su, A. Marcus, D. H. Raulet, M. C. Hammond, An RNA-Based Fluorescent Biosensor for High-Throughput Analysis of the cGAS-cGAMP-STING Pathway. Cell Chemical Biology 23, 1539-1549 (2016).
70. M. Su, J. Zheng, L. Gan, Y. Zhao, Y. Fu, Q. Chen, Second messenger 2′3′-cyclic GMP-AMP (2′3′-cGAMP): Synthesis, transmission, and degradation. Biochemical Pharmacology 198, 114934 (2022).
71. E. M. Kibby, A. N. Conte, A. M. Burroughs, T. A. Nagy, J. A. Vargas, L. A. Whalen, L. Aravind, A. T. Whiteley, Bacterial NLR-related proteins protect against phage. Cell 0 (2023).
72. T. Nugent, D. T. Jones, Transmembrane protein topology prediction using support vector machines. BMC Bioinformatics 10, 159 (2009).
73. E. F. Pettersen, T. D. Goddard, C. C. Huang, E. C. Meng, G. S. Couch, T. I. Croll, J. H. Morris, T. E. Ferrin, UCSF ChimeraX: Structure visualization for researchers, educators, and developers. Protein Science 30, 70-82 (2021).

### Tables

TABLE 1
Exemplary CD-NTase (Synthase) Enzymes of the Invention*

| CD-NTase | | |
|---|---|---|
| (Synthase) | SEQ ID NO. | Organism |
| 005-00 | (SEQ ID NO. 1) | Desulfotomaculum alkaliphilum |
| | | (DSM 12257) |
| 005-01 | (SEQ ID NO. 2) | Enterococcus faecalis (ATCC 29212) |
| 005-02 | (SEQ ID NO. 3) | Clostridioides difficile (QCD-76w55) |
| 005-03 | (SEQ ID NO. 4) | Bacillus thuringiensis (Bt407) |
| 005-04 | (SEQ ID NO. 5) | Lactobacillus brevis (BSO 464) |
| 005-05 | (SEQ ID NO. 6) | Clostridium botulinum (ATCC 17786) |

*Included in Table 1 are orthologs of the proteins, as well as polypeptide molecules comprising an amino acid sequence having at least 80%, 81%, 82%, 83%, 84%. 85%, 86%, 87%, 88%, 89%, 90%. 91%, 92%, 93%, 94%, 95%, 96%, 97%, 98%, 99%, 99.5% or more identity across their full length with an amino acid sequence of any SEQ ID NO listed in Table 1, or a portion thereof. Such polypeptides can have a function of the full-length polypeptide as described further herein.
*Included in Table 1 are RNA nucleic acid molecules (e.g., thymines replaced with uredines), nucleic acid molecules encoding orthologs of the encoded proteins, as well as DMA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid as described further herein.

What is claimed is:

1. A modified polypeptide that catalyzes biosynthesis of 2′3′-Cyclic-GMP-AMP (2′3′-cGAMP), wherein said modified polypeptide comprises an amino acid sequence having at least 70% identity to any one of the amino acid sequences according to SEQ ID NOs. 1-6, or a biologically active fragment thereof.

2. The modified polypeptide of claim 2, wherein the modified polypeptide catalyzes production of 2′3′-cGAMP in the absence of a ligand.

3. The modified polypeptide of claim 2, wherein the ligand is a double-stranded DNA.

4. The modified polypeptide of claim 1, wherein the 2′3′-cGAMP binds to and activates the Stimulator of Interferon Genes (STING) receptor.

5. The modified polypeptide of claim 4, wherein activation of STING occurs independently without the presence or activity of cyclic GMP-AMP synthase (cGAS).

6. The modified polypeptide of claim 1, wherein the polypeptide further comprising a heterologous polypeptide.

7. The modified polypeptide of claim 1, wherein the heterologous polypeptide is selected from the group consisting of a signal peptide, a peptide tag, a dimerization domain, an oligomerization domain, an antibody, or an antibody fragment.

8. The modified polypeptide of claim 7, wherein the peptide tag is a thioredoxra. Maltose-binding protein (MBP), SUMO2. Ghrtathione-S-Transferase (GST), calmodulin binding protein (CBP), protein C tag. Myc tag. HaloTag, HA tag, Flag tag, His tag, biotin tag, V5 tag, or OmpA signal sequence tag.

9. The modified polypeptide of claim 8, wherein the antibody fragment is an Fc domain.

10. The modified polypeptide of any one of claims 1-9, wherein the polypeptide is immobilized on an object selected from the group consisting of: a cell, a metal, a resin, a polymer, a ceramic, a glass, a microelectrode, a graphitic particle, a bead, a gel a plate an array, and a capillary tube.

11. A pharmaceutical composition comprising the modified polypeptide of any one of claims 1-9, and a pharmaceutically acceptable carrier.

12. A pharmaceutical composition of claim 11, wherein said pharmaceutically acceptable carrier selected from the group consisting of excipients, and/or diluents.

13. An isolated nucleic acid molecule encoding the polypeptide of any one of claims 1-9.

14. An expression vector comprising a nucleotide sequence, operably linked to a promoter encoding the nucleic acid of claim 13.

15. A host cell transfected with the expression vector of claim 14.

16. A method of producing a polypeptide comprising culturing the host cell of claim 15 in an appropriate culture medium to, thereby, produce the polypeptide and/or 2′3′-cGAMP.

17. The method of claim 16, wherein the host cell is a bacterial cell or a eukaryotic cell.

18. The method of claim 17, wherein the bacterial cell comprises a probiotic bacterial cell.

19. The method of claim 16, wherein the host cell is genetically engineered to express a selectable marker.

20. The method of claim 16, further comprising the step of isolating the polypeptide from the medium or host cell.

21. The method of claim 16, further comprising the step of isolating the 2′3′-cGAMP produced by the polypeptide from the medium or host cell.

22. A pharmaceutical composition comprising the 2′3′-cGAMP produced method of claim 21, and a pharmaceutically acceptable carrier.

23. A method of treating a disease or condition comprising administering a therapeutically effective amount of the pharmaceutical composition of claim 22 to a subject in need thereof, wherein the 2′3′-cGAMP activates the STING receptor in the subject.

24. A non-human animal model engineered to express a polypeptide of any one of claims 1-9.

25. The non-human animal model of claim 24, wherein the polypeptide is overexpressed.

26. The non-human animal model of claim 24, wherein the animal is a knock-in or a transgenic animal, and wherein the expression of cGAS has been disrupted or knocked-out.

27. The non-human animal model of claim 24, wherein the animal is a rodent.

28. A host cell genetically modified to express a nucleotide sequence encoding heterologous polypeptide that catalyzes biosynthesis of 2′3′-Cyclic-GMP-AMP (2′3′-cGAMP), wherein said polypeptide comprises an amino acid sequence having at least 70% identity to any one of the amino acid sequences according to SEQ ID NOs. 1-6, or a biologically active fragment thereof.

29. The host cell of claim 28, wherein the host cell is a bacterial cell or a eukaryotic cell.

30. The host cell of claim 29, wherein the bacterial cell comprises a probiotic bacterial cell.

31. A host cell genetically modified to express a nucleotide sequence encoding heterologous polypeptide that catalyzes biosynthesis of 2′3′-Cyclic-GMP-AMP (2′3′-cGAMP), wherein said polypeptide comprises an amino acid sequence having at least 70% identity the polypeptide of any one of claims 1-9, or a biologically active fragment thereof.

32. The host cell of claim 28, wherein the host cell is a bacterial cell or a eukaryotic cell.

33. The host cell of any of claims 28-32, and further comprising isolating the 2′3′-cGAMP produced by the polypeptide from host cell.

34. A pharmaceutical composition comprising the host cell of any of claims 28-32, and a pharmaceutically acceptable carrier.

35. A method of treating a disease or condition comprising, administering a therapeutically effective amount of the host cell of any of claims 28-34 to a subject in need thereof, wherein the 2′3′-cGAMP produced by the host cell activates the STING receptor in the subject.

36. A method of synthesizing 2′3′-cGAMP comprising contacting the polypeptide of any one of claims 1-9, or biologically active fragment thereof, with 2′3′-cGAMP nucleotide substrates.

37. The method of claim 39, wherein said 2′3′-cGAMP nucleotide substrates comprise adenosine triphosphate (ATP), and guanosine-5′-triphosphate (GTP).

38. The method of any one of claims 36-37, wherein contacting comprises the step of contacting in vitro, in vivo, or ex vivo.

39. The method of claim 38, wherein contacting comprises the step of contacting in a host cell.

40. The method of any one of claims 36-39, further comprising purifying the synthesized 2′3′-cGAMP.


SEQUENCE LISTING (6 sequences)

SEQ ID NO: 1 - protein, 455 aa, Desulfotomaculum alkaliphilum
MFDLETEFNIFYRDYVVLSKDEKQNLYNKKDLNLDRLKDGLQEYNEEKKTEYKIKDNVVQGSVAMSTVTQNDKHDYDIDVAVIFDKDNIPSGTTAVKNIVVNSLKKKCKQFKTEPEAKTNCVRVAYEEGYHIDFAVYRRFKNDSDEFEYEHCGSEWSKRDPRTITNWFIENNKAQDYKLRKIVRLLKMFCKSREHWVMPGGLIHTVLVVECFEPNDRIDKSFYNTIKATRDRLKNDKEVKNPVDDSLSLIIKESDKTKVENLYNRLSTYIDKLDILFTDGCTKEQAIEAWNDFFNHSYWSDLLTEDTQKANESAYCATETFPECDETEEFIEHIYPIDIKYDLNINCRVTQDGWRTKLLRSMLRLKEPLRLNKNLEFFIEGTNVPPPYKVFWKVRNIGDVAEQKNCIRGQIVEDKGKNTKKEETSFRGPHFVECYIVRYGVCVARSRIDVPINIL

SEQ ID NO: 2 - protein, 451 aa, Enterococcus faecalis
MYDCSKQLEKFYRKKVVLSAKEQNELREKRKLNIKRLKDGLSEYNSEKNKNYKIAEERIQGSMAMHTITQNDKQDYDIDVGIVFEEDSLNELGSLATRNMVANALERKTKQFAENPEIKTSCVRLKYNSTGYHVDFAVFKRYKKNSYDADYTYEHSGSEWSVRHIKDLESWFSERIKGKGDDLRKVIRLSKMFCKSRESWKNMPSGLIQTVICEENFNESYSRLDEIFYYTMKNIVRRLNVYLEVYAPVDNGRSLVNRDIDYQRMKNWKNRLETQLDSLNVLFDDECTSEEALKAWGIFFEHSYWNELANTVKEQNFTDTEEFIGDLYPVLEQFEVMVDCKVSGNGFTLMALQKYLEKYAPRLNKFIPPKFKIECSIKSTNCPSYDKILWKVLNVGSEAERRNEIRGQIKDRGKQITENTKFRGDHYIECYLIKDNICVGIGHVNVPIGGN

SEQ ID NO: 3 - protein, 453 aa, Clostridioides difficile
MFDLSIELKKFYYNEVVLSSEETKNLREKKKLNIKRLREGLSDYNADYNTNYAIAETLEQGSVAMSTVTQNEKNDYDIDVAIIFDKSNLNGLGPIAVKNVVADALKRKCTNFKTQPESKTNCVRIIYSDNYHIDFAIYRRIRNDDGSYNYEHAGSEWRPRDPRAINKWFRDEIKEHTEDLRKVVRLLKMFSKSRELWKMPGGLIQSVLSEEKIQNYKRMDELFFYTIREISVRLKKSIEVYNPTDDTQSLLLKQSDYKKMSNLCNRLENKLSALDILFDNECTKKDAVQAWYEIFNHDYWTYKEDENNMKFESFNKSFDTFEYDDTEEYIRNIVPVSNKYKITLDCRVIDKNGTIKKLSTIYNNGKSIPLNRKLLFYLDENRVPKPYDIYWKVKNEGVEAIERNCIRGQIFKNADPCFETIEERSDFVGNHYVECYIIKNGVCVAKDRIDVNI

SEQ ID NO: 4 - protein, 433 aa, Bacillus thuringiensis
MDMYNLSSKFNTFYNNHVVLQGEKQSELGKKKNTNIDRLKDGLKEYNDEHDTNYKLVDNVIQGSVAMHTVTQNDENEYDIDVAIIFDKSSIPDSTTRVKNIMVDALKRKCSGFRTEPEAKTNCIRIEYASGYHIDFAIYRRFLNDDGEYEYEHCGSEWRSRDPWAITNWFNTENTASNNDLRSVVRLLKMFCKSRDGWSMPGGLIQSVLASESVSSDARLDVTFYNTICNIRDRLGQDQEVYNPTNADLTLLYTVKDKQKVKNLHSRLTSYINKLSVLFEDDCTKEKAIEAWNDFFSHSYWAEQLPKNEERIQKTIDMFAEMTSAIGTQKETVTVQVFTELVSGQRIPLNAFKGKLPKGKPVTFIAEPSFTDYTDIKWFVNNTGDESVNNRSYDTNGKESTHETKYRGKHTVTCKVYRNQQVVCQTVKRVSVR

SEQ ID NO: 5 - protein, 452 aa, Lactobacillus brevis
MYDCSRNFNEFYHTKVILPEKSQNELRKKRKTNIKRLKSGLSEYNSENNTDFRISEERIQGSMAMHTVVQNDENDYDIDVGIVFESDNLDEIGPHAIKNILENALKRKMGQFAVEPLVKTSCVRMEYSNGYHVDFAIFKRSKIYLENEYTYEHAGAKWSTRHIKALEEWFNTETSKTNDNLRKIVRLSKMFCKSRKTWKNMPSGLIQTVLCDEAITNDYSRTDELFYYTMKKIVDRLNVNLEVNAPVDNERKLVIRDIDYTRVQNWKNRLENELTKLDVLFNPDCTFAEAVDAWGLFFNHSYWKELNMHETRKSLREDTQEVFNNTEEHIEDKYLVNEYYDVSINCKISGNGIRPMPIEKYLDSYFAKFIPHNFSVTCKIDHADCPPFDKVLWKVLNVGKESVKRNDIRGQILDRGEKITENTIFRGQHYIECYLIKNDICIAIGHVDIPIG

SEQ ID NO: 6 - protein, 469 aa, Clostridium botulinum
MYNLDKEIRSFYRKKVVLSSEKQKSLIEKKDLNLDRLKAGLKEYNEENGTDYKIVDTIIQGSVAMQTVIQAEDNNYDIDVGIAFNKDNLPQPITSENNTQKVKNIVHSALLKKATVFKFKPEKRTNCIRVIYSDGYHIDFAIYRRYKDENDEYIYEHCGADWVYRNPRTITKWFKSRDKAYNYNLKRIVRLLKAYLKQDSSWSVPGGLIQSVLADECFKSSSRLDEMLYNTIEAIVNRLEENTEVNNPVDKDSEGKEIPLLRTQEHKDKVIKMKNYIKKGLNKLSILFDGNECTEEKALRAWGDFFNNDYWTELADEISNTSIAESINKSFSALNQDVYDYNETEEFIEELYPINEKYNIDIECKVQQSGYTVDTLRNFLRFNKYLKPNKKLYFEVKNNTVPRPYKIYWKVLNAGNEAMKRNEIRGQIVKGDRTHTEDTLFSGNHKVYCYIVKNEECVAMNDIDVPIYI